(19) United States
(12) Patent Application Publication
(10) Pub. No.: US 2026/0148854 A1
(43) Pub. Date: May 28, 2026

(54) VERSATILE SYSTEM FOR PROVIDING CONTEXTUALIZED SUBJECT DATA AND ADAPTIVE GUIDANCE TO AN INTELLIGENT AGENT FOR DYNAMIC INTERACTION WITH A SUBJECT

(71) Applicant: GenoEmote LLC, Bellevue, WA (US)

(72) Inventor: Alexandrea Lauren Day, Bellevue, WA (US)

(73) Assignee: GenoEmote LLC, Bellevue, WA (US)

(21) Appl. No.: 19/366,192

(22) Filed: Oct. 22, 2025

Related U.S. Application Data
(63) Continuation-in-part of application No. 18/383,026, filed on Oct. 23, 2023, now abandoned.
(60) Provisional application No. 63/803,491, filed on May 9, 2025.

(51) Int. Cl. G16H 50/20; G06F 40/30; G16H 10/60

(52) U.S. Cl. CPC G16H 50/20; G06F 40/30; G16H 10/60

(57) ABSTRACT

The present disclosure details a versatile system for providing a digital system that evaluates one or more responses received from a subject of the system. The system provides a subject profile, comprising subject-specific historical, functional, clinical, or operational data relevant to the subject. A query engine provides one or more queries, or adaptive guidance, for the subject—and a digital agent is provided to interact with the subject in real time via a query or adaptive guidance from the query engine. One or more sensor devices communicate(s) or record(s) a response characteristic of the subject during interaction with the digital agent, and evaluation engine evaluates unified data based upon the subject's response to a query or adaptive guidance, in combination with the response characteristic.

### REFERENCE TO RELATED APPLICATIONS

This application claims priority to U.S. Provisional Ser. No. 63/803,491, which was filed on May 9, 2025, which is pending, and which is hereby incorporated by reference in its entirety for all purposes.

This application is a continuation-in-part application of and claims priority to U.S. Ser. No. 18/383,026, which was filed on Oct. 23, 2023, which is pending, and which is hereby incorporated by reference in its entirety for all purposes.

### TECHNICAL FIELD OF THE INVENTION

The present disclosure relates generally to the field of modular artificial intelligence-driven agentic systems that integrate natural language conversational agents with clinical-grade, wearable, non-contact biometric sensors, or other Internet of Things (“IoT”) networked or remote sensors. More specifically, the present disclosure relates to inventive systems, structures, and methods that provide an intelligent agent that ingests, validates, and contextualizes real-time or near-real-time behavioral, biometric, functional, and environmental data to guide subject interaction and decision-making workflows.

### BACKGROUND OF THE INVENTION

The technology market for precision medicine, digital therapeutics, and wellness is poised for widespread adoption and exponential growth, driven by rapid advancements in wearable technology and artificial intelligence (AI) powered healthcare solutions—such as agentic AI. Further growth in this market is also being fueled by public sector interest in increasing the role of “wearables” sensor devices in the form of watches, bands, rings, patches, headbands, earbuds, and clothes, as well as networked or remote IoT sensors that can be used for a variety of monitoring and measuring applications.

Conventional systems appear to offer certain aspects of agentic systems in conjunction with data received from sensor devices to follow a specific workflow. These systems typically report received sensor data to a subject's dashboard, in combination with some questionnaire or generic activity recommendation that lacks an engaging interaction with a subject. These conventional systems appear to lack a fully integrated workflow that is contextually aware of and evaluating sensor device data and subject input data, in conjunction with pattern recognition, to provide a real-time, responsive, and adaptive workflow that fully engages with a subject.

Similarly, the technology market for IoT devices and sensors has, in many aspects, already achieved widespread adoption—even as it continues to grow rapidly. Typical IoT systems appear to offer certain aspects of receiving data from remote sensor devices communicatively connected to another IoT monitoring device or dashboard. These conventional systems often follow a specific workflow without real-time adaptation or contextual evaluation of sensor data. Any interaction with a subject generally takes the form or reporting a certain value or displaying a dashboard. Any interaction with the subject to change the data shared with the subject usually requires the subject to change some setting or element in the connected device. Similarities between these conventional systems and conventional digital therapeutic systems extend to an apparent lack of a fully integrated workflow that is contextually aware of sensor device data and subject input data, or pattern recognition between the two. As such, conventional systems fail to provide real-time, responsive, and adaptive guidance that fully engages with a subject.

For example, in clinical remote patient monitoring “RPM”) applications, conventional platforms stream vitals (BP, SpO2, HR/HRV, weight, glucose) to dashboards and trigger fixed-threshold alerts. Human staff must then call or message patients to learn the “why” behind the alert—causing delays and driving alert fatigue due to constant human intervention. Such conventional interactions are retrospective rather than event-timed, and such systems rarely capture contemporaneous patient explanations (e.g., “just climbed stairs,” “pain episode”) or link them with contemporaneous measurements. Rules are mostly static (having limited hysteresis and minimal personalization), workflows are manual, and provenance is fragmented.

In the context of wearable, consumer, and clinical-grade sensor devices (e.g., watches, rings, patches), such devices stream steps, HR/HRV, sleep, and sporadic BP/SpO2 into dashboards that issue generalized tips (e.g., “move more,” “sleep earlier”). These systems seldom prompt at the moment a threshold deviation occurs, rarely elicit subject context (e.g., “just drank coffee,” “post-workout”), and do not link that context to contemporaneous signals to deliver brief, subjected training. Recommendations remain static and retrospective, leaving subjects uncoached in real time, and limiting lasting behavior change.

In the context of decentralized clinical trials (“DCT”), symptoms and adverse events-capture often rely on static, recall-based ePRO forms completed hours or days after onset, while data from wearable devices and sensors sits in dashboards. Administrators and staff must chase context by phone, adding delay and burden. In the context of post-acute care, hospital at home platforms stream vitals and trigger fixed alerts, also requiring staff to chase context.

As such, using conventional systems and platforms to capture timely, consistent vitals and intake data that complement clinical workflows is difficult, because processes are staff-dependent in treatment rooms or areas, and largely impractical to do remotely, causing delays and variability.

Recognizing these and other limitations of conventional systems, there is therefore a need for a versatile system—comprising a multitude of constructs, methods, and resources—for providing an intelligent agent with contextualized real-time subject data, and adaptive guidance, for agent interaction with a subject.

### SUMMARY OF THE INVENTION

The system of the present invention addresses the shortcomings and limitations of prior solutions. The system of the present invention identifies, comprehends, and solves numerous problems that appear to have been previously unrecognized and/or unaddressed. The system of the present invention provides an intelligent agent with contextualized real-time or near real-time subject data, and adaptive guidance, for decision-making workflows and agent interaction with a subject.

The present invention recognizes that there is a need for RPM systems that proactively prompt a subject for context data at the moment of an adverse event or threshold deviation, record subject-provided context, reconcile the data with reference settings, and adapt guidance/escalation in real time while reducing human involvement.

The present invention recognizes that there is a need for systems that transform wearable device and sensor streams into event-timed, context-aware, personalized, coaching with adaptive guidance and follow-ups, rather than dashboard summaries.

The present invention recognizes that there is a need for DCT that, upon detecting a deviation (e.g., HR/SpO2 change, pain spike), proactively prompt participants in the moment to capture a brief explanation, link it with contemporaneous signals, and log provenance—enabling real-time, context-aware symptom reporting, adherence support, and risk-based escalation.

The present invention recognizes that there is a need for “hospital at home” systems that, on parameter deviation (e.g., BP rise, wound-area temperature change), proactively prompt patients for a brief explanation, link it with contemporaneous signals (e.g., activity, meds, timing), and log provenance—enabling real-time triage and next-step guidance, with only clinically actionable cases routed to human clinicians.

The present invention recognizes that there is a need for systems that enable guided, validated self-capture with brief context across settings—at home pre-visit, in the treatment room before the appointment, and at emergency department arrival. This means intake can occur wherever the patient is with their data reaching clinicians as a structured, triage-ready bundle aligned to existing workflows.

The present invention recognizes that there is a need for systems that provide in-the-moment living guidance based upon networked devices, smart home systems, or emerging virtual assistants. There is also a need for systems that, when multi-signal patterns from such devices shift, briefly engage a subject for determining context, linking any reply with contemporaneous signals, and log provenance (e.g., lock reminder, lights off, quiet mode, meds packed, route suggestion, safety check).

The system of the present invention provides collaborative and analytical constructs that dynamically identify patterns or trends in subjects and data received from subjects. An intelligent agent of the present invention assesses, and guides subjects based on real-time behavioral, functional, physiological, and contextual inputs. The agent detects patterns, validates responses, and dynamically adapts subject interaction flow based on evolving subject states. The interaction flow may be delivered via text, voice, or a digital avatar—which may take the form of a digitally created agent, the form of an image or video of human agent—depending on the client's preference. In certain embodiments, the interaction flow may also comprise a true human agent—being advised or guided by a digital agent. The system of the present invention accumulates data from each subject interaction and develops a longitudinal record of subject behavior and risk.

The present disclosure details a versatile digital system that evaluates one or more responses received from a subject of the system. The system may comprise a reference database for a selected population or control group, which may comprise, for example, response characteristics and conditions, device settings, and operational parameters. The system of the present inventions comprises a subject profile, comprising subject-specific information or data—gathered in a specific interaction or longitudinally over time—relevant to contextualization of one or more responses received from the subject.

The system present invention comprises a query engine that presents the subject with one or more queries retrieved from a database of predetermined queries or directions. The query engine may simply retrieve predetermined queries or directions, or it may dynamically or iteratively adapt or generate queries and directions.

The system of the present invention further comprises an intelligent, digital agent that interacts with the subject in real time using queries or directions; and one or more sensor devices, adapted to communicate or record a response characteristic of the subject while the subject is responding to interaction from the digital agent. As used in the present disclosure, a response characteristic may comprise any sensor data or reading taken from or by a given sensor device.

The system of the present invention further comprises a contextualization engine—which may comprise a pattern recognition module—that evaluates a response characteristic of the subject with respect to data from either the subject profile or the reference database, or both. The system of the present invention may also comprise an adaptive guidance module that revises—or creates new—queries or directions for a subject based upon analysis from the contextualization engine. All of the constituent parts of the digital system are communicatively or operationally connected to each of the others.

The present disclosure details a versatile system for providing a digital system that evaluates one or more responses received from a subject of the system. Such responses may be provided in a number of forms, depending on the specific embodiment. For example, a subject response may be verbal or textual in form, take the form of tapping a button, or take the form of performance of an assigned activity (e.g., putting on a wearable sensor to measure blood pressure). Numerous other variations are comprehended by, and fall with the scope of, the present invention.

The system provides a subject profile, comprising subject-specific historical, functional, clinical, or operational data relevant to the subject. A query engine is provided for presenting one or more queries, or adaptive guidance, to the subject—and a digital agent is provided to interact with the subject in real time via a query or adaptive guidance. One or more sensor devices communicate(s) or record(s) a response characteristic of the subject during interaction with the digital agent, and contextualization engine evaluates the subject's response to a query or adaptive guidance, in combination with the response characteristic.

The present invention further provides a digital system for evaluating one or more responses received from a subject of the system. The system comprises a subject profile, comprising subject-specific historical, functional, clinical, or operational data relevant to the subject. A query engine is provided that presents one or more queries, or adaptive guidance, to the subject. The system further comprises a digital agent that interacts with the subject in real time via a query or adaptive guidance. One or more sensor devices communicates or records a response characteristic of the subject during interaction with the digital agent. The system further comprises a contextualization engine that evaluates the subject's response to a query or adaptive guidance, in combination with the response characteristic.

The present invention further provides a digital clinical evaluation system for evaluating one or more responses received from a human subject of the system. The system comprises a subject profile, comprising subject-specific historical, diagnostic, clinical, or observational data relevant to the subject. A query engine is provided that presents one or more queries to the subject; and a digital agent interacts with the subject in real time via a query. The system further provides one or more sensor devices that communicates or records a response characteristic of the subject during interaction with the digital agent. A contextualization engine evaluates the subject's response to a query, in combination with the response characteristic.

Other features and advantages of the present disclosure will be apparent to those of ordinary skill in the art upon reference to the following detailed description taken in conjunction with the accompanying drawings.

### BRIEF DESCRIPTION OF THE DRAWINGS

For a better understanding of the invention, and to show by way of example how

the same may be carried into effect, reference is now made to the detailed description of the invention along with the accompanying figures in which corresponding numerals in the different figures refer to corresponding parts and in which:

FIG. 1 illustratively depicts certain aspects of a digital system according to the present invention;

FIG. 2 illustratively depicts certain aspects of a processing unit according to the present invention;

FIG. 3 illustratively depicts certain aspects of a digital system according to the present invention;

FIG. 4 illustratively depicts certain aspects of a digital system according to the present invention;

FIG. 5 illustratively depicts certain sensor devices according to the present invention;

FIG. 6 illustratively depicts certain aspects of a digital system according to the present invention;

FIG. 7 illustratively depicts certain aspects of a digital system according to the present invention;

FIG. 8 illustratively depicts certain aspects of a digital system according to the present invention;

FIG. 9 illustratively depicts certain aspects of a digital system according to the present invention;

FIG. 10 illustratively depicts certain aspects of a digital system according to the present invention;

FIG. 11 illustratively depicts operational aspects of a digital system according to the present invention;

FIG. 12 illustratively depicts certain aspects of a digital system according to the present invention;

FIG. 13 illustratively depicts certain aspects of a digital system according to the present invention;

FIG. 14 illustratively depicts certain aspects of a digital system according to the present invention;

FIG. 15 illustratively depicts operational aspects of a digital system according to the present invention; and

FIG. 16 illustratively depicts operational aspects of a digital system according to the present invention.

### DETAILED DESCRIPTION

While the making and using of various embodiments of the present disclosure are discussed in detail below, it should be appreciated that the present disclosure provides many applicable inventive concepts, which can be embodied in a wide variety of specific contexts. The description hereinafter details a number of illustrative embodiments of the present invention's system for contextualizing a subject's response to a query based upon reference and sensor data. The illustrative embodiments and topologies described herein are, however, merely examples of a variety of ways to make and utilize the disclosed invention—and they are not presented in an order or manner that should be construed to limit the scope of this disclosure. Quite the opposite is true, in fact. There are numerous variations and embodiments that—with the benefit of this disclosure—are enabled for those of skill in the art.

A number of operational constructs, elements, and/or components are provided by the present invention to address the limitations of prior approaches and to present innovative systems and operations that go well beyond the scope of previous technology. Unless otherwise specifically indicated otherwise, these constructs, elements, and/or components may be provided as independent components or segments, as components or segments of a larger system, or as varied combinations of both. All such constructs, elements, segments, and/or components may communicate and/or interoperate with other constructs, elements, and/or components. Various aspects of the present invention may take the form of hardware implementations, an entirely software implementation, or an implementation combining software and hardware aspects. Even where additional or alternative embodiments are described or illustrated, this disclosure comprehends further variations that are not explicitly described or depicted.

In various examples, a component or element may comprise some form of artificial intelligence (“AI”) construct—such as a rule-based module, a machine-learning regressor, a machine learning classifier, a neural network, generative or non-generative operation units, any combination thereof. These are examples only for illustrative purposes, and it should be understood that other constructs and combinations are comprehended by the present invention.

To the extent that embodiments of the present invention comprise or utilize AI constructs or operations, those embodiments are novel, non-obviousness to a person of ordinary skill in the art, possess practical utility and provide tangible benefits, and fall-under 35 USC § 101—into recognized categories of patentable material.

The AI in the embodiments disclosed and claimed herein fall within the one of the four statutory categories: processes, machines, manufactures, or compositions of matter. Such embodiments further comprise more than mere mathematical concepts, methods of organizing human activity, and mental processes. Some claims merely involve or may be based on certain ideas, but do not require or explicitly site such ideas.

To the extent that any embodiments disclosed and claimed herein do incorporate a recognized abstract idea, such embodiments further integrate those aspects to improve the functioning of a computer or computer system, and do not merely link such an idea to a particular technological environment. Many such embodiments disclosed and claimed herein comprise and require: application-specific circuits or devices having specific hardware components; systems for monitoring patients or other subject entities using specific sensor hardware and data processing; or AI-driven treatment methods with specific medical applications. In fact, many of the embodiments disclosed and claimed herein are directed specifically to AI-driven treatment methods with specific medical applications.

Other embodiments disclose and claim the integration or use of any number of remote sensor technologies—such as biometric and environmental sensors. There are certain embodiments that disclose a truth or accuracy detection module, that may evaluate biometric sensor data from non-verbal communication from a subject experiencing a certain condition (e.g., an emotion) while connected to biometric sensors. In other embodiments, such a module may evaluate sensor data gathered from other methods—such as affect evaluation, facial expression analysis, or voice analysis. All such data, whether affirmative responses only or data from non-affirmative responses, may be recorded or stored for longitudinal or baseline subject profiles, or any other evaluation components of the present invention.

A “Gene expression measurement”, as used in this disclosure, is usually achieved by quantifying levels of the gene product, which is often a protein. Two common techniques used for protein quantification comprise Western blotting and enzyme-linked immunosorbent assay or ELISA Note, new methods of measuring proteins and other substances, cells, electrical impulse etc., are being discovered and developed, and so such new technologies are comprehended by the present invention.

A “health status recording and reporting system”, as used in this disclosure, relates to a component or subsystem that maps a personality trait or condition to a disease condition. Such a mapping system may comprise a digital framework, including a pattern recognition module, that assesses commonly experienced emotions and relates certain beliefs and diagnosed or reported disease conditions of a subject. This particular health status recording and reporting system gathers and analyzes query responses from a subject, in combination with sensor/device data and—depending upon the embodiment—compares those to reference data, thereby providing health-status updates with time stamps and source information. This system may comprise an adaptive guidance module that modifies query/interaction flows based upon this unified data.

A “health status”, as used in this disclosure, may comprise mental health, physiological health, fitness health, or machine health. In certain embodiments, mental/physiological health status may comprise: any commonly experienced emotion, belief, and diagnosed or reported disease conditions of a subject; a prediction of disease conditions; or personality risk factor. Further, a health status, as used in this disclosure comprises four domains: (i) mental health status (emotions, beliefs, mood, motivational state); (ii) physiological health status (vitals, signs, diagnosed or reported conditions, risk) (iii) fitness status (readiness, fatigue, recovery, load tolerance); and (iv) machine status for related devices/assets (e.g., wear, overload, fault condition). Each status may be directly measured or inferred, and is stored as a current estimate (and, when useful, a short-term outlook) with strength and confidence. This may be evaluated or measured at the moment a change is detected, combining the reply with the readings, and routing the result to guidance, triage, or improving workflows.

Aa used in this disclosure, the terms “real time” or “real-time” may mean instantaneous, or as close to instantaneous as possible considering slight lags in time introduced due to data transmission hardware, software, media, or protocols.

An “unconscious agenda”, as used in this disclosure, means perceptions, beliefs, mindsets, or latency states of a control group of subjects selected from a population of subjects.

A “therapist”, as used in this disclosure, means and comprises a human or digitally manifest therapist, or chatbot, avatar, coach/friend. These are merely examples, and do not limit the scope of the present invention.

A “computer”, as used in this disclosure, means any machine, device, circuit, component, or module, or any system of machines, devices, circuits, components, modules, or the like, which are capable of manipulating data according to one or more instructions—including, for example, a computer processor, a microprocessor, a central processing unit, a general purpose computer, a super computer, a personal computer, a laptop computer, a palmtop computer, a notebook computer, a desktop computer, a workstation computer, a server, or the like, or an array of computer processors, microprocessors, central processing units, general purpose computers, super computers, personal computers, laptop computers, palmtop computers, notebook computers, desktop computers, workstation computers, servers, or the like.

A “server”, as used in this disclosure, means any combination of software and/or hardware, including at least one application and/or at least one computer to perform services for connected clients as part of a client-server architecture. The at least one server application may comprise, but is not limited to, an application program that can accept connections for service requests from clients by sending back responses to the clients. The server may be configured to run the at least one application, often under heavy workloads, unattended, for extended periods of time with minimal (or no) human direction. The server may comprise a plurality of computers configured, with the at least one application being divided among the computers depending upon the workload. For example, under light loading, the at least one application can run on a single computer. However, under heavy loading, multiple computers may be required to run the at least one application. The server, or any of its computers, may also be used as a workstation.

A “database”, as used in this disclosure, means any combination of software and/or hardware, including at least one application and/or at least one computer. The database may comprise a structured collection of records or data organized according to a database model, such as, for example, but not limited to at least one of a relational model, a hierarchical model, a network model, or the like. The database may comprise a database management system application (DBMS) as is known in the art. The at least one application may comprise, but is not limited to, for example, an application program that can accept connections to service requests from clients by sending back responses to the clients. The database may be configured to run the at least one application, often under heavy workloads, unattended, for extended periods of time with minimal human direction. Depending upon the embodiment, there are a number of database types that may be provided in accordance with the present invention. For example, a distributed database may be provided. In other embodiments, a dynamic database may be provided as the ML components of the present invention require.

A “communication link”, as used in this disclosure, means a wired and/or wireless medium that conveys data or information between at least two points. The wired or wireless medium may comprise, for example, a metallic conductor link, a radio frequency (RF) communication link, an Infrared (IR) communication link, an optical communication link, or the like, without limitation. The RF communication link may comprise, for example, WiFi, WiMAX, IEEE 802.11, DECT, 0G, IG, 2G, 3G, 4G, 5G, 6G, or subsequent cellular standards, Bluetooth, and the like.

The terms “including”, “comprise” and variations thereof, as used in this disclosure, mean “including, but not limited to”, unless expressly specified otherwise.

The terms “a”, “an”, and “the”, as used in this disclosure, mean “one”—whereas a “plurality” means “more than one”—unless expressly specified otherwise.

The terms “construct,” “engine,” “component,” “subsystem” and “module”—as used in this disclosure, may be used interchangeably and mean any operable hardware, software, or combinations of hardware and software, that are provided and configured to function or operate to deliver particular outputs based upon particular inputs.

Devices, components, modules, or other subsystems that are in communication with each other need not be continuously communicating with each other, unless expressly specified otherwise. In addition, components, modules, or other subsystems that are in communication with each other may communicate directly or indirectly through one or more intermediaries.

Although operations such as process steps, method steps, or algorithms may be described in a sequential order, such operations may be alternatively configured to different orders. Unless otherwise specifically described as requiring a certain order, such operations may be performed in any order. Further, some steps may be performed simultaneously. All such variations are comprehended by the present invention.

A “computer-readable medium”, as used in this disclosure, means any medium utilized in providing data (for example, instructions) which may be read by a computer. Such a medium may take many forms, including non-volatile media and volatile media.

In addition to storing code and/or data on machine-readable storage media, typical electronic devices can transmit and/or receive code and/or data over one or more machine-readable transmission media (also called a carrier) (e.g., electrical, optical, radio, acoustical or other forms of propagated signals-such as carrier waves, and/or infrared signals). For instance, typical electronic devices also comprise a set of one or more physical network interface(s) to establish network connections (to transmit and/or receive code and/or data using propagated signals) with other electronic devices. Thus, an electronic device may store and transmit (internally and/or with other electronic devices over a network) code and/or data with one or more machine-readable media (also referred to as computer-readable media). Wired transmission media may comprise coaxial cables, copper wire, and fiber optics, including the wires that comprise a system bus coupled to the computer processor.

The terms “user,” “therapist,” “provider,” or “administrator”—which may be used interchangeably hereafter—refer to an entity (e.g., an individual person, an electronic system, a separate AI agent) that prompts the system of the present invention to generates a query or queries via an electronic device. Software and/or services may use credentials to distinguish different accounts associated with the same and/or different users. Users can have one or more roles, such as administrator or programmer/developer. As an administrator, a user typically accesses electronic devices to administer them for other users, and thus an administrator often works directly and/or indirectly with server devices and client devices.

As used in the present disclosure, the term “contextualization” means some form linking, comparing, relating, or merging sensor derived data with some other reference data source. Contextualization may therefore be used to mean merging a certain sensor data with responses from a subject. Contextualization may further be used to mean comparing certain sensor data with data from a pre-populated database. Other variations and combinations may be user throughout this disclosure without departing from the scope of meaning for this term.

As used in the present disclosure, the terms “validation” or “validating” mean some form of evaluating one element of data against a second, reference element of data, to determine if the one element of data meets a certain predefined criteria. For example, the one element of data may be evaluated to determine if it is in a proper format, meets a predetermined threshold, or lies within an expected range. In some instances, validation may utilize or encompass contextualization. Other variations and combinations may be user throughout this disclosure without departing from the scope of meaning for this term.

The terms “subject,” “client,” or “patient”—which may be used interchangeably hereafter—refer to an entity (e.g., an individual person, an electronic system) that receives or responds to queries from the system of the present invention via an electronic device. Software and/or services may use credentials to distinguish different accounts associated with the same and/or different subjects. Subjects may refer to human subjects, or other devices that operate as a client, or sensor, for other devices or operations.

Embodiments of the present invention may be directed to health psychology—with focus on correlating specific emotions, and the latency states that underlie them, to specific disease conditions. The terms “unconscious agenda” and “latency state” may be used interchangeably throughout the present disclosure and generally refer to an operational, psychological, or emotional condition that influences subject behavior, decision patterns, or communication style but is not explicitly articulated by the subject. A latency state may therefore be considered as an underlying, not-directly-observable condition that influences behavior or performance, inferred from observed or reported evidence. For humans, this may be considered a “latent motivational state” (e.g., subconscious motivations or triggers) such as avoidance, threat sensitivity, reward seeking). In applications where a subject is an electronic device or system, latency state may be considered as “latent operational state” (e.g., fault conditions, erroneous code segments) that affects performance despite normal status readings.

The system of the present invention reveals (or surfaces) insights not otherwise available to a therapist or client and improves communication of latent information. It relates unconscious agendas (e.g., latent motivational states) to health outcomes and adapts interactions in real time. The same pattern extends to clinical care and remote monitoring, athlete readiness, and machine/vehicle status (deriving momentary context from statements or telemetry and focusing human attention on actionable events).

The present invention thus provides insights not available to either a user or a subject (at work, home, or at play) and improves communication and unconscious information-sharing. The system of the present inventions analyzes the relationship between latency states and disease conditions. In certain embodiments, the system includes a latency-state modeling subsystem that enables an agent to identify, monitor, and respond to latent agendas exhibited by a subject during interactions.

According to the present invention, an agent may perform multi-modal data analysis, including linguistic tone, response hesitation, topic recurrence, sentiment polarity shifts, and biometric or behavioral inputs, to infer potential latency states. These inferences are represented as structured and quantifiable latent variables (e.g., avoidance, resistance, dependency, defensiveness, or suppressed affect) within the agent's internal context model. Throughout the framework of the present invention, an agent may support psychiatric assessment and therapeutic engagement by detecting and adapting to unconscious subject dynamics, thereby enhancing accuracy, empathy, and therapeutic alignment in a digital mental health application.

In certain embodiments, for example, if the subject interacting with an agent repeatedly diverts from emotionally charged subjects or displays abrupt topic changes, the agent may infer a latency state of avoidance and adapt its interaction strategy by introducing reflective questioning or adjusting the pacing of emotional exploration. In another example, a latency state of dependency may be inferred when a subject exhibits recurring requests for reassurance, prompting the agent to reinforce autonomy-supportive interventions.

A latency-state modeling subsystem according to the present invention may be integrated with a clinical safety and validation layer, requiring human clinician oversight for significant behavioral interpretations or interventions. All inferred latency states are logged with provenance data and confidence scores, ensuring traceability and compliance with clinical validation protocols.

Embodiments of the present invention further provide adaptive guidance, that may be directly communicated to a subject or embedded within other directions to or communications with a subject. As used in this disclosure, adaptive guidance means delivering subjected prompts or control actions to influence a subject's latent state toward a desired condition. The system of the present invention may then observe the effect(s) of such interactions, and iteratively adjusting subsequent actions based on the measured response.

Depending on the embodiment, the present invention's adaptive guidance may comprise some form of belief or behavioral modification or reprogramming. As used in the present disclosure, adaptive guidance (or “behavioral reprogramming”) is a term that generally refers to methods aimed at modifying, redirecting, or conditioning patterns of thought, function, operation, emotion, or action—and may occur consciously or subconsciously. Depending on the scientific, therapeutic, or operational context, the present invention comprehends a number of alternatives, analogs, or forms that may be provided to achieve (or augment) adaptive guidance. These other forms may be considered in the context of psychological, neurological, physiological, functional, and computational domains.

In embodiments that are provided in a clinical or therapeutic context, these other forms may focus on altering cognitive or emotional processing through structured intervention or self-directed learning. For example, certain embodiments may provide Cognitive-Behavioral Restructuring (“CBR”)—which is modification of maladaptive thought patterns that drive unwanted behaviors. Other embodiments may provide conditioning or counterconditioning—which comprise classical and operant conditioning techniques that reinforce or extinguish specific behavioral responses. Other embodiments may provide schema therapy or modification, in which deeply ingrained cognitive and emotional schemas that influence behavior are reworked.

In embodiments addressed to neurological or physiological adaptive guidance, systems or modules may be provided for neuroplastic conditioning; biofeedback (or neurofeedback); pharmacological modulation; or deep brain (or Vagus nerve) stimulation. Various alternatives to, or combinations of the above, are all comprehended by the present invention.

In embodiments of the present invention addressed to adaptive guidance for computational or AI-driven systems, systems or modules may be provided for behavioral reinforcement modeling; adaptive interaction flow adjustment; agentic state realignment; or digital habit reconfiguration systems. Again, a number of additional or alternative forms, or various combinations thereof, are all comprehended by the present invention and all fall within its scope.

In certain embodiments, adaptive guidance may be provided to redirect or dispel unwanted or inhibiting beliefs—providing access to a subject's subconscious with AI, machine learning (“ML”), or sensors. For example, currently available biometric sensors may be used to measure the changes in the pupil (such as pupil dilation) to detect a subject's inaccurate or deceptive response, at a rate of 80-86% accuracy. The present invention provides far more extensive biometric analysis comprising one or more sensor devices to reach a 99.99% accuracy rate.

Throughout the framework of the present invention, an agent may support psychiatric assessment and therapeutic engagement by detecting and adapting to unconscious subject dynamics, thereby enhancing accuracy, empathy, and therapeutic alignment in digital mental health application.

Various embodiments of the present invention provide pre-established and expanding databases or repositories accessible to select and present queries to a subject. In some embodiments, reference data comprises digitized emotional parameters and latency states, which the system digitizes for use in subject interactions or evaluations. In other embodiments, reference data may comprise reference settings (e.g., textbook ranges, model specs) and a subjects learned, sensor-derived baseline profile. A reference profile may be initialized from norms (e.g., 98.6° F. temperature, model RPM bands) and adapted over time from validated signals (e.g., seizure pattern, HRV, unit-specific RPM) and may be provided to interpret inputs and set thresholds. In other embodiments, reference data may comprise “baseline data” gathered from generalize populations, studies, or control groups.

In certain embodiments, the system of the present inventions provides digitized emotional parameters to enhance contextual responsiveness and behavioral adaptation of an AI agent. Digitized emotional parameters may be represented as structured data elements derived from physiological, behavioral, or linguistic inputs, such as tone of voice, facial expression, typing cadence, or sentiment analysis of textual content. These parameters are converted into normalized emotion vectors (e.g., confidence, frustration, engagement, satisfaction) that the agent uses to dynamically adjust interaction strategies.

For example, an agent may detect a reduction in subject engagement (e.g., prolonged response latency or negative sentiment tone) and automatically adjust its dialog style, response pacing, or content complexity to re-engage the subject. In another example, a support agent monitoring real-time voice and text inputs may identify elevated stress levels and initiate empathy-mode responses, including simplified explanations, reduced query intensity, or escalation to human assistance.

Digitized emotional data may be stored in association with session records, enabling longitudinal analysis of subject's sentiment trends or adaptive learning models that refine agent behavior over time. The emotion recognition subsystem may further enforce operational boundaries by ensuring that emotional inference data are processed within subject-consented scopes and are not used for unrelated profiling or decision-making.

In this manner, the systems of the present invention provide emotion-aware agent and AI behavior, allowing agents to simulate empathy, maintain subject trust, and improve outcome quality through adaptive, contextually informed interactions.

Embodiments of the present invention further provide rule sets, which govern various operational aspects of the system. These rulesets map conditions to query type, timing, and modality so interactions are consistent across clinical, fitness, and machine-status contexts. For example, a rule set may be provided for governing the types of queries that are presented to a subject.

Still other embodiments of the system of the present invention provide add-on modules for both interaction and education, such as: interaction modules that layer human-tempered behaviors (e.g., empathy cues, culturally aware phrasing, adaptive pacing with brief pauses, and interleaved micro-queries triggered by contemporaneous inputs) into any workflow; and education or training modules that teach users and subject how emotions and latent motivational states relate to health and performance—and how to describe them during interactions —across domains such as clinical RPM, athlete readiness, ambient/wearable contexts, and machine/asset operations. Such modules may be selectable per domain and configurable per user—improving flow, comprehension, and quality of captured context, while preserving timestamps, provenance, and auditability.

Embodiments of the present invention provide the system with subject-specific information relevant to the specific workflow of the system. For example, subject-specific information of a patient and/or person providing answers to questions may comprise a list of diagnosed disease conditions, personal stories, defined traumas, aspirations, preferences, inherent or acquired belief systems, session notes, and/or observation(s) by others. Other embodiments of the present invention may provide athlete data (e.g., sport/role, training phase, injury history, RPE/wellness logs); machine/asset data (e.g., IDs, configuration, tolerances, maintenance logs, telemetry); driver/vehicle data (e.g., telematics, gaze/blink measures, recent trip context); and ambient/wearable context (e.g., location, sleep/activity, air quality, calendar). Each type of data may be sourced from appropriate data storage or databases (e.g., EHR, CMMS, telematics, wearable platforms) for use during query interactions.

Some embodiments provide machine learning technology that assesses data gathered from these various embodiments to differentiate between true, false, or “don't know” replies from a subject in response to queries. Such machine learning technology may be initialized or trained with example data sets and periodically revised and calibrated by outside resources (e.g., human evaluator)—and governed by rule sets or models generated by human subject matter experts. Such machine learning technology is not the present invention in and of itself. Rather, the technology is one of a number of analytic tools incorporated into embodiments of the present invention to provide data processing, pattern recognition, and evaluation capabilities that are not possible by humans.

In embodiments of the present invention, communication between a system user, a subject, and a machine learning engine may be provided in any suitable manner. For example, certain embodiments may convert voice to text for processing. In other embodiments, a machine learning engine may be trained to ingest conversational communication. Various embodiments may comprise: machine learning or natural language processing techniques; latent semantic indexing; latent Dirichlet allocation; word or sentence embedding models; collaborative filtering techniques; entity graphs; Jaccard similarity; and cosine similarity or translation models.

In certain embodiments of the present invention, software or hardware may provide an improved virtual embodiment experience in the metaverse or other digital environments. Other embodiments may provide a language agnostic environment with attributes common to human or brain interaction, such as: gestures; expressions; movements; emotions; beliefs; intent; and intuition. In such embodiments, these attributes may be implemented in the form of a life-like virtual agent—incorporating haptics to transmit emotions, intuition, and intent.

Embodiments of the present invention implemented in a metaverse, or other digital, environment adds security to the agent by virtue of validating or invalidating responses to queries-thus protecting against exploitation. While the exterior of the agent may change its visual form (i.e., a morphing avatar), the identity of the avatar itself (the owner) is fixed and known only to the owner unless the owner chooses to share it. The agent's unique properties allow its persona to build trust and choose authentic friends—and distinguish whether others are safe to trust. This agent of the present invention may be used between one virtual agent to another, as well as human to virtual agent. Thus, certain embodiments of the present invention provide generation of a 3D virtual agent that is capable of representing a real person, in the way they communicate and feel.

Other embodiments of the present invention may provide combined software and hardware configured as a guidance system that adapts to epigenetic signals. For example, when an unfavorable epigenetic profile is recognized, the system of the present invention may adjust prompts, thresholds, and follow-ups, and schedule targeted interventions according to defined safety and provenance controls.

Epigenetics is the study of gene expression. Epigenetics is a method to analyze changes in DNA expression to determine what emotions and behaviors are related to disease conditions. DNA or chromosomal evaluation data may be ingested by systems of the present invention to determine what interventions or regimens provide the highest probability for successfully and therapeutically altering belief and behavior patterns of a subject and is a validation method chosen, among others. Reference and correlation data detailing or describing causation links between a subject's genetics and the issues that they are presenting with may be evaluated by an algorithm or other analytical engine, including AI constructs. This data is then analyzed in relation to clinically desirable or therapeutic belief or behavior modalities.

According to the present invention, embodiments of such modalities may be as simple as relaxation, breathing and repeating of de-programming and reprogramming statements. Statements may be confirmed as effective or correct by analysis of biometric sensor data in combination with: personal information of the subject, a reference database storing generalized data concerning emotions and unconscious beliefs, and AI rule sets.

The reference database may comprise volumes of data concerning physical and mental disease conditions, and correlated emotions and beliefs. This data may be compiled from external sources from data concerning specific populations or control groups. In a number of alternative embodiments, reference data may comprise general operating data for a certain type of system or device. An independent subject-specific database or profile may comprise patient personal information such as the subject's personal history, personality traits and preferences, traumas throughout life, aspirations, record(s) of latency states, reported or diagnosed disease conditions, and therapy session notes (if applicable).

Various embodiments of the present invention may provide a unified reference profile stored within the reference database. Depending upon the embodiment, a reference profile of the present invention may comprise a unified baseline comprising: normative reference data that applies to a general population or control group (e.g., textbook ranges, model specs, “standard” clinical data); and subject-specific accumulated or sensor-derived baseline date. The reference profile may be initialized from established or well-known baselines or norms (e.g., 98.6° F., model RPM bands) and supplemented or adapted over time based upon validated signals (e.g., seizure pattern, HRV, unit-specific RPM). Depending upon the embodiment, such a reference profile may be used in interpreting inputs and setting thresholds.

In various embodiments, biometric sensor data may comprise detection of eye and/or facial movements, pulse, respiration, blood pressure, heart-rate variability, temperature, electrodermal activity, brain waves, and/or DNA/epigenetic signals. Generalized, non-biometric, sensor data may comprise network appliance data related to the subject's performance or behavior, or data retrieved from other “internet of things” (“IOT”) systems in use for the subject. The system of the present invention may also comprise a variety of other sensor technologies, such as RF/proximity IDs; RFID/NFC readers; Bluetooth beacons; Wi-Fi; position/rotation; GPS; gyroscopes; microphones; and cameras.

For purposes of the present disclosure, it should be noted and understood that the term “therapist” is not used in a limiting sense. In other embodiments of the present invention, a user querying a subject may be anyone from a coach to a parole officer, or anyone in between. Hereinafter, all such possibilities are referred to as “analyst(s).” Similarly, “subject” may refer to a patient or client, an application subject, a network device, another digital agent, an IoT system, or any other subject type that is being queried. All such terms may be used interchangeably, depending on the specific context of a particular embodiment.

The present invention is described in greater detail now with specific reference to the drawing figures. It should be clearly understood that the embodiments in these drawing figures are provided for illustrative purposes, but in no way are intended to limit the scope of the present invention. Upon reference to the present disclosure, the drawing figures, and the illustrative embodiments therein, those of skill in the art will be enabled to practice not only the embodiments depicted, but also numerous other embodiments of the present invention that are not explicitly illustrated. All such embodiments are, however, comprehended by the present invention and fall within the intended scope of the present disclosure.

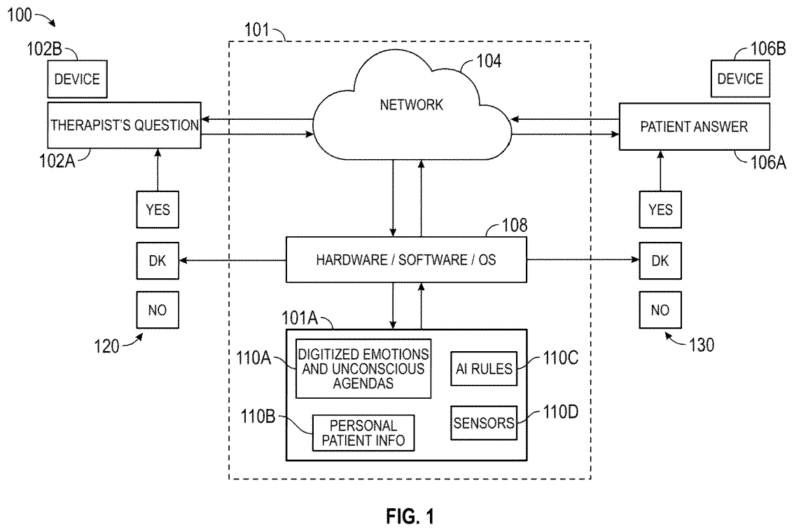

Certain aspects of the present invention are now described in general reference to system 100, as depicted in FIG. 1. System 100 comprises a digital system that evaluates one or more responses received from a subject of the system. In the embodiment depicted, system 100 is provided as a health status recording and reporting system comprising a digital framework 101.

Framework 101 comprises an operational data construct 101A that aggregates, and centralizes access to, a variety of data sources used in the operation of system 100. Construct 101A comprises a reference database 110A (or other suitable data repository), a subject profile 110B, a rules database 110C (or other suitable data repository), and one or more sensors 110D. In the embodiment depicted, database 110A comprises a library of digital data, models, and other hardware processable abstractions, that characterize and represent emotions and latency states of a control group or a generalized population. In other embodiments, database 110A may comprise digital data, models, and other hardware processable abstractions, that characterize and represent other characteristics (e.g., performance or functioning) of control groups or generalized populations of humans, or non-human systems.

Subject profile 110B is a database, or other suitable data repository, for recording or storing subject-specific historical, functional, clinical, or operational data concerning the subject. Such data may comprise longitudinal data collected from subject querying, sensor device readings, or imported data from other sources. In the embodiment depicted, database 110B comprises patient profile and personal information which may comprise (but is not limited to): electronic health records (“EHR”), a recode of diagnosed disease conditions, personal history, defined traumas throughout life, aspirations, personality traits and preferences, the patient's belief systems, record of discovered latency states, reported diagnosed disease conditions, therapeutic or other treatment notes, and/or observation by other humans expressed in reports.

Rules database 110C is a database, or other suitable data repository, that provides rules and guidelines for querying and interaction with a subject. Depending upon the embodiment, the rules and guidelines may comprise regulatory compliance data, rules, and operational parameters for analytical or AI constructs throughout system, educational regimens or data related to subject interactions, communication parameters for subject interactions, and interpretive parameters.

In the embodiment depicted, database 110C may comprise functional and operational rules related to: education and training in regard to emotions, latency states, disease conditions of a control group of subjects; communication models for understanding responses of a control group of subjects; a human-tempered response framework; interpretation of questions posed to a control group of subjects; or interpretation of answers of the control group of subjects.

Each sensor 110D is communicably and operationally coupled to framework 101 (via construct 101A), and to a subject. Each sensor 110D communicates, records, or reports a physical, functional, or operational state (or characteristics thereof) of a subject during querying of the subject.

In the embodiment depicted, each sensor 110D is communicably and operationally coupled to framework 101, via construct 101A, during a patient question-answer session with a therapist (or chatbot, avatar, coach or friend, etc.), and is configured to communicate, record, or report a physiological or emotional state (or characteristics thereof) of a patient while responding to a question posed by the therapist during the question-answer session.

Framework 101 further comprises a network interface module 104 that is operatively and communicatively coupled to construct 101A via an infrastructure or operations engine 108. Engine 108 provides hardware and/or software components that provide or perform framework 101's operations—such as a processor or other hardware executing or running an operating system (“OS”), software applications, software application programming interfaces (API's), software modules, virtual machines, and runtime libraries, for example. Although engine 108 may be a functionally or physically integrated subsystem of framework 101, embodiments of the present invention comprehend that engine 108 may not be so integrated (e.g., distributed allocation of component bandwidth shared with other systems and subsystems.

Depending on the embodiments, aspects of module 104 may provide a distributed storage platform or network. Other aspects of module 104 may provide a communicative and operational interface to a user device 102B and a subject device 106B. In embodiments where module 104 provides a distributed storage network, module 104 may comprise a distributed database application or a distributed ledger (e.g., blockchain) application. Blockchain technology may be provided to manage data using a distributed, secure network architecture. Data stored in a blockchain cannot be easily compromised. Therefore, data that is deemed or considered sensitive may be securely stored in a blockchain system, to prevent data corruption and unauthorized access thereto.

Even though blockchain and distributed ledger technology has become somewhat ubiquitous, and implementation of that technology should be understood upon reference to the present disclosure to those of skill in the art, the following is provided as an example embodiment of the present invention. Distributed storage network 104 may be implemented as a blockchain application used to process and store data securely within a distributed storage environment—using a peer-to-peer network and Public Key Infrastructure (PKI) cryptography.

Alternatively, in other embodiments of the present invention, network 104 may be implemented as a distributed database application (e.g., common applications used in big data platforms and cloud computing platforms) that processes and stores data securely within a distributed storage environment.

The distributed storage platform may also be provided as a combination of a block chain application and a distributed database application. Data stored in the distributed storage environment may comprise (without limitation) optimization variables, data models, or sensor and control variables. In certain embodiments, a copy, digital twin, or a subset of such data may be stored in the cloud so that any implemented AI/ML systems may be executed more efficiently.

System 100 further comprises communicative and operational connections to a user device 102B and a subject device 106B, via module 104. The connection to user device 102B provides access to, and interaction with, a user of system 100 that intends to query or interact with a subject. The connection to subject device 106B provides access to, and interaction with, a subject of system 100 for interaction with or querying of that subject. System 100 is agnostic as to the specific form or features of devices 102B and 106B, as long as the connections to those devices are configured to transmit data to, and receive data from, framework 101. In certain embodiments, device 102B may comprise a mobile device, a server, an IoT device or system, or a computer through which a user of system 100 interacts with the system. Other device types, and various combinations thereof, are all comprehended by the present invention. Similarly, certain embodiments of device 106B may comprise a mobile device, a server, an IoT device or system, or a computer through which a subject of system 100 interacts with the system. Other device types, and various combinations thereof, are all comprehended by the present invention.

Functionally or operationally, framework 101 receives a user query 102A via device 102B. Framework 101 may adapt, supplement, modify or otherwise process query 102A before transmitting the query to subject device 106B. In other embodiments, framework 101 may transmit the query without any processing. In certain embodiments, query 102A may be processed by engine 108 before transmission to subject device 106B. For example, in situations where devices 102B and 106B utilize different operating systems, engine 108 may perform a simple conversion of query 102A into a format compatible with device 106B before transmitting to device 106B. Other variations of processing may similarly be performed by engine 108 prior to transmission of query 102A, and all such variations are comprehended by the present invention.

In other embodiments, for example, engine 108 may provide pre-transmission processing of query 102A by providing processing that query with or through construct 101A (or one of its constituent components). For example, engine 108 may process query 102A by accessing data from database 110A to reference data from database 110A in query 102A. In certain embodiments, this processing may be provided based upon reference to database 110C, profile 110B, or data from sensors 110D. All variations and combinations thereof are comprehended by the present invention.

In other embodiments, engine 108 may provide pre-transmission processing of query 102A by accessing an AI construct governed by database 110C. In various embodiments, this processing may be provided based upon reference to database 110A, profile 110B, or data from sensors 110D. All variations and combinations thereof are comprehended by the present invention.

Similarly, other embodiments of the present invention may comprise engine 108 performing pre-transmission processing of query 102A by accessing profile 110B, or data from sensors 110D—with or without reference to the other constituent components of construct 101A. All variations and combinations thereof are comprehended by the present invention.

Functionally or operationally, framework 101 receives a subject response 106A (to query 102A) via device 106B. Framework 101 may adapt, supplement, modify or otherwise process response 106A before transmitting the response to user device 102B. In other embodiments, framework 101 may transmit the response without any processing. In certain embodiments, response 106A may be processed by engine 108 before transmission to subject device 106B. For example, in situations where devices 102B and 106B utilize different operating systems, engine 108 may perform a simple conversion of response 106A into a format compatible with device 102B before transmitting to device 102B. Other variations of processing may similarly be performed by engine 108 prior to transmission of response 106A, and all such variations are comprehended by the present invention.

In other embodiments, for example, engine 108 may provide pre-transmission processing of response 106A by processing that query with or through construct 101A (or one of its constituent components). For example, engine 108 may process response 106A by accessing data from database 110A to reference data from database 110A in response 106A. In certain embodiments, this processing may be provided based upon reference to database 110C, profile 110B, or data from sensors 110D. All variations and combinations thereof are comprehended by the present invention.

In other embodiments, engine 108 may provide pre-transmission processing of response 106A by accessing an AI construct governed by database 110C. In various embodiments, this processing may be provided based upon reference to database 110A, profile 110B, or data from sensors 110D. All variations and combinations thereof are comprehended by the present invention.

Similarly, other embodiments of the present invention may comprise engine 108 performing pre-transmission processing of response 106A by accessing profile 110B, or data from sensors 110D—with or without reference to the other constituent components of construct 101A. All variations and combinations thereof are comprehended by the present invention.

In certain embodiments, framework 101 may provide query, prompt, or response specific data 120 via a user interface transmitted from framework 101 to device 102B, or query, prompt, or response specific data 130 via a subject interface transmitted from framework 101 to device 106B.

As an example, user 102B and subject devices 106B may provide access to a great variety of contextually specific data, including separate user interfaces with different data available, that may comprise a data set exclusively for the user making a query or a data set exclusively for the subject making a response to a query. Such data may be locally stored on engine 108 or network 104.

In the specific embodiment depicted in FIG. 1, system 100 is provided as a health status recording and reporting system. System 100 comprises a digital framework 101 that provides a pattern recognition module 101A, configured to record and report health status of a patient subject. Digital framework 101 comprises: (1) a digitally recorded library 110A of human emotions and latency states of a control group of subjects selected from a population of patient subjects; (2) a digitally recorded set of rules 110C related to at least one of: (i) education and training in regard to emotions, latency states, disease conditions of the control group of patient subjects, (ii) communication models for understanding, responses of the control group of patient subjects, (iii) a human-tempered response framework, (iv) interpretation of questions posed to the control group of patient subjects, and (v) interpretation of answers of the control group of patient subjects; and (3) a digitally recorded patient profile 110B of the subject, where the patient profile comprises personal information of the patient, personal information comprises a list of diagnosed disease conditions, personal life stories, defined traumas throughout life, aspirations, personality traits and preferences, the patient's belief systems, record of discovered latency states, reported diagnosed disease conditions session notes, and/or observation by other humans expressed in reports.

System 100 comprises one or more sensor devices 110D communicably connected to framework 101 and to the patient during a question-answer session with a clinician or therapist—which may be human, a chatbot, an animated or static avatar, or may be a coach or a trusted friend. Each sensor 110D may provide communication, recording, or reporting of at least one of: a physiological state or an emotional state of the patient while responding to a plurality of questions posed by the therapist during a question-answer session. Digital framework 101 provides integration of a first input from the digitally recorded library 110A of human emotions and latency states, a second input from the digitally recorded set of rules 110C, and a third input from the digitally recorded subject profile 110B of the subject, a fourth input from a sensor 110D, and a fifth input about a physical or a mental disease condition. Digital framework 101 further validates accuracy of the patient's response to the plurality of questions posed by the therapist, based on the first input, the second input, the third input, the fourth input and the fifth input. The accuracy of the patient's response to the plurality of questions posed by the therapist comprises a statistical level of confidence score, calculated based on data collected from the control group or general population of patients. Framework 101 further maps and predicts a possible disease condition of the patient, based on the first input, the second input, the third input, the fourth input and the fifth input—wherein the possible disease condition may comprise a medically diagnosed disease condition. Framework 101 further displays or reports the predicted disease condition of the patient on a display, in a file, or on a visible or printable report.

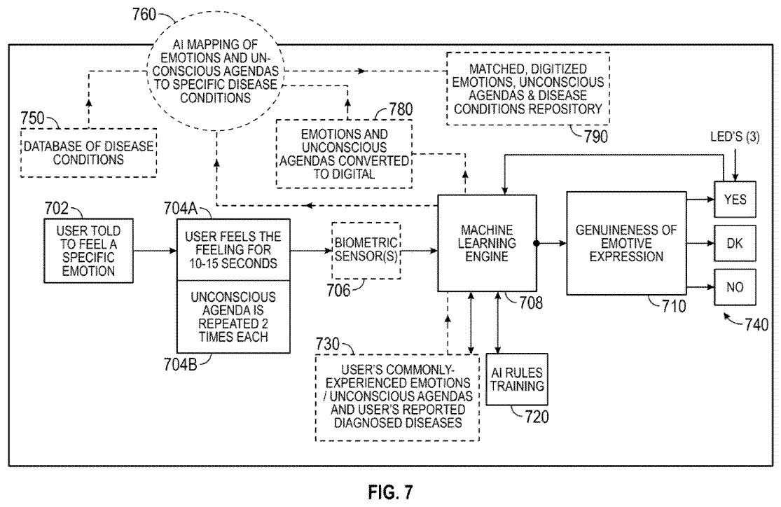

The set of rules is used by the therapist or another clinician or technician to train framework 101 to ask questions based on a list of emotions or latency states. Framework 101 comprises a second digitally recorded library of physical and mental disease conditions (as described in greater details with reference to FIG. 7, hereinafter), that may be correlated with emotions and beliefs systems of the patient. This correlation may be provided as an output of an AI or machine learning (“ML”) subsystem, or other pattern recognition subsystem, that identifies (for example) a correlation between patients reporting a diagnosed disease and those patients also reporting frequently experienced specific emotions and beliefs.

A database or repository of commonly felt emotions and latency states may be derived or formed from patients using system 100 to report their experiences or, alternatively, may be imported from other data sources having similar data content.

In one embodiment, module 101A may comprise or perform statistics-based pattern recognition (e.g., stochastic modeling techniques), and may comprise AI subsystem, that predicts a disease condition based on a similarity score or metric, representing estimated similarity between a control group of patients'reported disease conditions with commonly felt emotions or latency states from the same control group. In one embodiment, module 101A comprises an ML module, trained (based upon the rules provided) to translate a patient's responses to a plurality of digitally recorded questions, together with a plurality of outputs received from the sensors, into actionable identifications, predictions, or characterizations concerning the patient's disease state or latency state(a).

Throughout the present invention, a variety of AI subsystems may be provided to implement or otherwise conduct functional or operational needs of system 100. As used in this disclosure, AI systems provide computational frameworks designed to perform tasks such as pattern recognition, decision-making, natural language processing, and predictive analytics. In most instances, such tasks are traditionally considered to be within the scope and capability of human cognitive function. Increasingly, however, such systems and tasks now require or utilize vast amounts of data that is well beyond the ability of human cognitive function to fully process or understand. Similarly, pattern recognition using AI systems is capable of finding correspondences and correlations between details in data that are simply imperceptible by humans. Thus, the present invention incorporates AI constructs as a building block of the overall invention and, where utilized, the AI constructs implemented perform at levels exceeding human capability.

As used in the present disclosure, AI systems or subsystems may comprise an input layer and data preprocessing module. An input layer receives raw or structured input data, such as numerical vectors, images, text sequences, or sensor readings. A preprocessing module normalizes, scales, or transforms that data, to enhance compatibility with subsequent layers.

As used in the present disclosure, AI systems or subsystems may comprise a computational model. According to the present invention, the computational model may comprise machine learning (“ML”) model, that may be provided as a neural network. As used in the present invention, neural networks comprise interconnected layers of nodes (also known as “neurons”), where each node applies a weighted sum of inputs followed by a non-linear activation function.

The present invention may provide a neural network comprising: a Feedforward Neural Network (“FNN”); a Convolutional Neural Network (“CNN”); a Recurrent Neural Network (“RNN”) or variants thereof; or a Transformer Model (“TM”). Common variations, combinations, or derivatives of such neural networks are all comprehended by the present invention.

As used in the present disclosure, FNNs comprise an input layer, one or more hidden layers, and an output layer, with data flowing unidirectionally. Each connection between nodes has an associated weight parameter, and the hidden layers enable feature extraction through hierarchical representations. CNNs are provided for processing matrix or grid-like data and comprise convolutional layers that apply: filters (e.g., “kernels”) to detect local patterns; pooling layers for dimensionality reduction (e.g., “max-pooling”); and fully connected layers for classification.

As used in the present disclosure, RNNs (and variants thereof) are provided for processing sequential data (e.g., time series, text)—with loops allowing information persistence across time steps. The present invention comprehends providing Long Short-Term Memory (“LSTM”) units or Gated Recurrent Units (“GRUs”) to address vanishing gradient issues by incorporating gates (e.g., forget, input, output) to regulate information flow.

As used in the present disclosure, Transformer Models (“TMs”) provide natural language processing and beyond. According to the present invention, TMs comprise self-attention mechanisms to weigh input token importance dynamically. These structures may comprise encoder-decoder stacks, positional encodings to maintain sequence order, and multi-head attention layers, where attention scores are computed as functions of query, key, value matrices, and d_k dimension of keys.

As used in the present disclosure, AI systems or subsystems may comprise an Output Layer and a Post-Processing Module. The Output Layer is a final layer that generates predictions, such as class probabilities. The Post-Processing Module may provide thresholding, non-maximum suppression (in object detection), or ensemble methods combining multiple models.

As used in the present disclosure, AI systems or subsystems may be provided via specialized hardware, including: Graphics Processing Units (GPUs) for parallel matrix operations, Tensor Processing Units (TPUs) for optimized tensor computations, or edge devices for real-time inference. Memory hierarchies (e.g., RAM for model weights, cache for activations) may be provided to ensure efficient data handling.

In machine learning-centric embodiments of the present invention, constructs may be parameterized by millions to billions of trainable weights, and stored in tensors, to approximate complex functions from data.

As provided in the present disclosure, AI systems, particularly those based on machine learning, may be provided with an inference phase—where a trained model processes new inputs to produce outputs. This phase may comprise forward propagation of input data through network layers. For a neural network, this comprises matrix multiplications and activations. This phase may further comprise feature extraction and representation learning, which may comprise hidden layers configure to automatically learn hierarchical features. In TMs, self-attention computes contextual embeddings, providing a model that captures long-range dependencies without recurrence.

As provided in the present disclosure, an inference phase may comprise inference optimization. To enhance efficiency, techniques such as quantization (e.g., reducing weight precision from 32-bit float to 8-bit integer) or pruning (removing low-importance weights) may be applied. In real-time embodiments, batch processing or asynchronous execution on distributed systems ensures low latency. According to the present invention, the inference phase may further comprise error handling and robustness constructs. In certain embodiments, operational safeguards may be provided—such as input validation to prevent adversarial attacks (e.g., perturbations that mislead the model). The system of the present invention may further comprise uncertainty estimation via Bayesian neural networks, which model weight distributions rather than point estimates. As such, the present invention provides machine learning in such a way that the system may generalize from training data to unseen examples-distinguishing it from rule-based AI, which relies on hardcoded logic without adaptation.

According to the present invention, training of AI systems and subsystems is critical to machine learning technology as described herein—and where a model iteratively adjusts parameters to minimize prediction errors on a dataset. This process is data-driven and optimization-based—and may provide the following operations and structures for the process.

Various embodiments of the present invention may provide data preparation for training the system. Training requires a labeled or unlabeled dataset. In supervised learning (e.g., classification, regression), inputs are paired with ground-truth outputs. In unsupervised learning (e.g., clustering via k-means or autoencoders) identifies patterns without labels. Reinforcement learning provides an agent interacting with an environment, receiving rewards to maximize cumulative returns via policies like Q-learning.

Various embodiments of the present invention may further provide a loss function definition—which is a scalar objective that quantifies model performance and identifies losses. Such losses may comprise: Mean Squared Error (“MSE”) for regression; cross-entropy for classification: −Σ y_i log(ŷ_i), promoting confident predictions.

Various embodiments of the present invention may further provide, in relation to training the system, an optimization algorithm where parameters are updated using gradient descent variants, and backpropagation computes gradients via a chain rule. Certain embodiments may provide Bayesian Optimization. Other embodiments may provide Stochastic Gradient Descent (SGD) to process mini-batches, or advanced optimizers like Adam that incorporate momentum and adaptive learning rates with bias-corrected updates.

Various embodiments of the present invention may further provide, in relation to training the system, a training loop and regularization. Over time, the model provided by the present invention iterates through a dataset, updating weights. Regularization techniques are provided to prevent overfitting, including L1/L2 penalties, dropout (i.e., randomly deactivating neurons during training, or early stopping based on validation loss. Other embodiments may provide transfer learning wherein pre-trained models (e.g., BERT for NLP) are fine-tuned on domain-specific data, initializing weights from bodies or groups of data.

Various embodiments of the present invention may further provide, in relation to training the system, evaluation and hyperparameter tuning. In such embodiments, metrics like accuracy, precision-recall, or an F1-score assess performance on held-out test sets. Hyperparameters (e.g., layer count, learning rate) are optimized via grid search, random search, or Bayesian optimization.

According to the present invention, embodiments comprising large-scale ML, distributed training across clusters (e.g., using data parallelism) accelerates convergence, with frameworks managing gradient aggregation.

In various embodiments, the system of the present invention may provide one or more front-end interaction modules configured to capture, interpret, and transmit subject input into structured workflows. These modules may take the form of: an avatar, representing a visual or conversational interface for human subjects; an agent, representing an autonomous or semi-autonomous task-oriented process aligned with a playbook; or a Language Understanding (“LU”) model.

As used herein, the term “LU model” refers to a specialized natural language processing component configured to receive unstructured human language input and generate structured representations of intent, entities, or semantic meaning. The LU model operates as a core interpretive layer within the system architecture—enabling downstream modules (e.g., agents, avatars, or workflow orchestration engines) to process and act upon subject-provided language inputs with precision.

In certain embodiments, an LU model functions independently or in conjunction with higher-capacity large language models. An LU model may be trained on domain-specific corpora to improve accuracy in extracting intents and entities relevant to a particular system function. In other embodiments, the LU model interfaces with dialog agents or avatars, providing semantic parsing and disambiguation to ensure that subject inputs are constrained to validated playbook boundaries.

An LU model may further integrate with validation gateways, dependency management systems, or agent orchestration engines. Such integration ensures that interpretations generated by the LU model are verified against scope rules, tracked with provenance metadata, and prevented from propagating into organizational knowledge without human or automated validation.

Each embodiment of a front-end interaction module may provide the primary interface layer of the system. For example, in an avatar-based implementation, the system presents an animated or conversational digital persona that directly interacts with the subject. In an agent-based implementation, interaction occurs through task-specific dialog flows aligned with organizational functions. In an LU model-based implementation, the LU model operates as the interpretive gateway, converting raw subject inputs into structured semantic representations for downstream orchestration. These and other variations and combinations are all comprehended by the present invention.

Upon reference to this disclosure, those of skill in the art will realize and appreciate that there are numerous applications of system 100. System 100 may comprise an application where the subject is an athlete or client, and the user is a coach or trainer. System 100 may comprise an application where the user is a clinician, and the subject is a recipient of an advanced technology prosthesis that relies upon the clinician to monitor, update, or calibrate settings of the prosthesis remotely. System 100 may comprise an application where the user is a processing system residing in centralized location that remotely maintains, troubleshoots, and updates IoT connected monitors, detectors, cameras, gauges, or other similar devices, without human involvement (unless there needs to be an escalation). These and a variety of other end use applications are all comprehended by the present invention and fall within the scope of the invention.

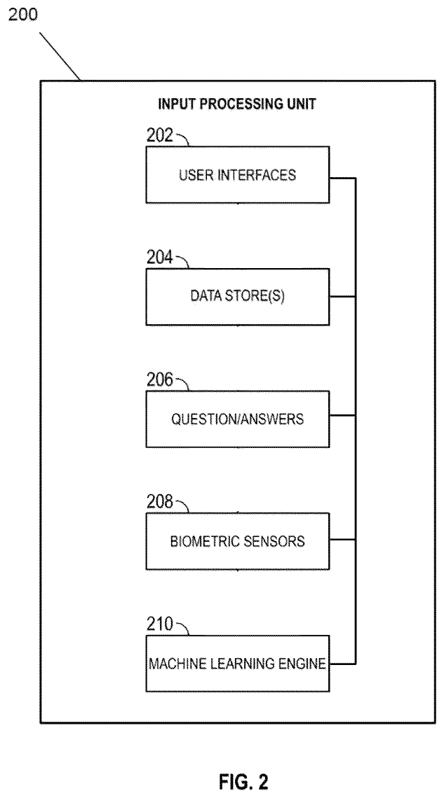

Referring now to FIG. 2, one embodiment of an input processing unit 200 according to the present invention is depicted. In this and other similar embodiments, unit 200 is provided to create (or aid in the creation of) an AI assistant to a user of system 100 via framework 101. Depending upon the specifics of any given embodiment, such an AI assistant may be alternatively or additionally provided to or for a subject via framework 101.

In the embodiment depicted, unit 200 comprises one or more input processing components or modules—accessible by a user or a subject via a user interface 202 on either's device. Unit 200 comprises an operational engine 210. In the specific embodiment depicted in FIG. 2, engine 210 comprises a machine learning engine.

Responsive to receiving a query or a prompt from a user via interface 202, engine 210 may access data from a data store (i.e., database) 204. Data 204 may comprise, among other things, a knowledge base of information pertaining to the field that system 200 is being used within. In the specific embodiment depicted in FIG. 2, the knowledge base comprises physical and mental health related data. Data 204 may further comprise personal information of the subject, as collected or retrieved by interface 202 on the subject device. Engine 210 may poll or search data 204 to retrieve, analyze, or otherwise process identified query and response data.

In some embodiments, such analysis may comprise comparing one or more characteristics related to the subject, or to already accumulated data concerning the subject. In the specific embodiment depicted, such characteristics may comprise (but not be limited to): traits, attributes, events, specific unconscious agendas or emotions, biometric sensor signals, responses, demographic data (e.g., age, gender, location), behavioral data, query techniques used, stylistic content (e.g., style, diction, tone, voice, intent, sentence/dialogue length and complexity), and psychographic data (e.g., opinions, values, attitudes, tempered responses).

In such embodiments, a subset of the characteristics may be provided to a scoring or comparison algorithm/model for evaluation. The scoring or comparison algorithm/model may generate and/or assign scores or labels to the evaluated characteristics. The scoring or comparison algorithm/model may use the generated scores/labels to determine a similarity score or metric, queries, or responses. The similarity metric may represent the estimated similarity between current and prior queries/responses. specific questions/answers or responses. In such embodiments, the resulting processed or customized data may be provided to create, organize, populate, or update a machine learning engine for a specific query/response related to a disease condition.

In other embodiments, characteristics related to a subject or already accumulated data concerning the subject may comprise different data. Such data may comprise (but not be limited to): device-specific data (e.g., manufacturer, model, generation, firmware or software revision, operational condition(s), damage conditions), subject performance data, queries or requests received from a subject, and alerts sent or received. As described above, a scoring or comparison construct may generate and/or assign scores or labels to the evaluated characteristics and render a similarity metric with respect to a preferred data set. Upon reference to the present disclosure, those of skill in the art will realize and appreciate that numerous variations and combinations of data may be provided in accordance with the present invention.

In other embodiments, engine 210 may be provided to access one or more data sources and/or application programming interfaces (“APIs”). In some embodiments, engine 210 may access one or more data sources comprising logic for composing one or more queries to solicit information from a subject. Information obtained as a result of presenting the query may be obtained and processed accordingly.

A ML model may apply decision logic to determine a hierarchal data traversal process for collecting and analyzing provider query (or question), and subject response (or answer), data. In certain embodiments, question/answer component 206 may associate one or more established rule sets (or models) to facilitate the deployment and/or implementation of an AI Therapy Assistant, and a rule set (or model) to one or more computing devices, services, or user accounts.

In another embodiment, input processing unit 200 may generate or create a morphing avatar, as described herein. The morphing avatar creation provided by input processing unit 200 may comprise the operations and inputs previously described in relation to system 100. In alternative embodiments, a single system—comprising one or more components such as computer processor and/or memory—may perform the methods and processes described in relation to systems 100 and 200.

In this embodiment, unit 200 may comprise user interface 202, data store(s) 204, index generation engine 206, and biometric sensors 208. Interface 202 may be provided to receive, store, and provide access to content—such as human characteristics or morphing avatar components for one or more avatars or agents. In such embodiments, interface 202 accesses various data sources comprising human characteristics relating to one or more avatars or agents. Such data sources may comprise photos and videos renderings of multiple different combinations of genders and races, behavioral data, and biometric sensor 208 interactions. The collected data may be stored by a data store accessible to interface 202, such as data stores 204. Data store(s) 204 are provided to store and/or organize data according to various criteria. For example, data store(s) 204 may store photos and videos, human characteristic data, colors, colors matched to words, meanings of words, emotions, or intent.

Index engine 206 may create a personalized index generation engine. For example, engine 206 may receive a request to generate a persona index. The request may be associated with one or more specific combinations for an avatar or agent with regard to gender or race. A request may be transmitted to engine 206 via interface 202—or received directly via an interface component accessible by a client or client device. In response to receiving such a request, engine 206 may access biometric sensor data 208 collected by interface 202 and/or stored by data store(s) 204. In this example, engine 206 searches for and collects data associated with the one or more specific persona or agents identified in the request. The morphing aspects associated with the one or more specific persona or agents (“personalized data”) may be combined with a persona index (or a generic persona index) and processed to create of a personalized persona index (e.g., a persona index corresponding to the personalized data for the specific avatar/entity). Processing the personalized data may comprise identifying and categorizing biometric data 208.

Processing personalized data may comprise determining and categorizing conversation data associated with persona/agents identified in the request. For example, determining similarities between a specific avatar/entity and another avatar/entity (e.g., the “other person”) in a metaverse setting may comprise machine learned techniques, natural language processing techniques, and/or sentiment analysis, to analyze and compare morphing aspects of the “other person.” Such analysis/comparison may comprise latent semantic indexing, latent Dirichlet processing, word and/or sentence embedding models, collaborative filtering techniques, entity graphs, Jaccard similarity, cosine similarity, and/or translation models—such as color coding and decoding. Such an analysis/comparison may further comprise validation indicators. In at least one example, the analysis may comprise comparing one or more characteristics, such as stylistic data (e.g., style, diction, tone, voice, intent, sentence/dialogue length and complexity, etc.) or color and shape assignments to emotions, intent, words or the meaning of words, or gestures, movements, or facial expression.

In such an embodiment, at least a subset of the characteristics may be provided to a scoring or comparison algorithm/model for evaluation. The scoring or comparison algorithm/model may generate and/or assign scores or labels to the evaluated characteristics. The scoring or comparison algorithm/model may use the generated scores/labels to determine a similarity score or metric for any form of avatar/entity. The similarity /ore/ metric may represent the estimated similarity between a specific avatar/entity and the other person/entity. In such embodiments, processed personalized data may be used to create, organize, populate, or update a personalized persona index for the avatar/agent identified in the request.

Engine 206 may be provided to access one or more conversational data sources and/or APIs. For such embodiments, engine 206 may access one or more data sources comprising remote or metaverse data. Remote or metaverse data may be used to supplement data in a persona index. Color-coded and color decoded data may comprise morphing aspects and human characteristics collected/derived from a plurality of subjects, and relating to one or more personas/agents, events, time periods, and/or conversational scenarios. Such conversational data may comprise conversational algorithms/models for processing with the biometric sensors 208 and the morphing aspects of the avatar/agent. In such embodiments, conversational data may be collected from the metaverse or other setting and stored in a metaverse chat index. The metaverse chat index may comprise metaverse subjects'perceptions, opinions and knowledge, their intentions, emotions, thoughts, and feelings regarding actions, communications and/or events relating to one or more specific avatars/agents, a period of time, or one or more events.

For example, metaverse engagement may be two-way with subjects interacting and learning from each other and coupled with machine learning advance future communications especially when enhanced by biometric sensors 208 collecting and exchanging information between two subjects, each with an interface 202, connected to the index generation engine 206 and receiving analyzed and converted data and language from biometric sensors 208.

In other embodiments, engagement is one-way and only one subject interface 202 is immersed in the metaverse. Signals, analysis, and conversion of language are received by that one subject. The one subject can still hear the words, meaning of words, and convert them into color and decode upon receipt. This provides the subject with the ability to hear any language not understood and have it understood upon conversion or translation from color to words and meaning of words, in real-time.

Engine 206 may generate an avatar or agents or LU model. In certain embodiments, for example, unit 200 may cause engine 206 to generate one or more avatars or agents (or instances thereof). Unit 200 may then cause or facilitate the application of data from a persona index to the one or more generated avatars or agents. Applying personalized data to an avatar or agents may generate a personalized avatar or agents, to interact conversationally in the persona of a specific avatar/entity.

In embodiments in which a subject creates more than one avatar, the algorithm will identify these two avatars as one virtual agent in order to not disturb model 208 when comparing/finding similarities between avatars. Applying personalized data to an avatar or agent may also cause a voice font, or a 3D model of an avatar/entity, to be applied to the avatar or agents. Engine 206 may further establish a set of interaction rules for an avatar or agents—such as emotion, facial expression, intent, movement or any other expression of thought or feeling.

Depending on the embodiment, a set of interaction rules may comprise determining when (and in what order) to utilize data and various data sources available to engine 206. As an example, engine 206 may establish a rule set dictating that, in response to receiving dialogue input, a specific avatar or agents may a response using data from the following data sets: 1) morphing aspects from a specific person/entity; 2) morphing aspects from subjects similar to the specific person/entity; 3) morphing aspects from a global subject base that may or may not be similar to the specific person/entity; and 4) generic, catch all phrases/questions that are not specific to the specific person/entity.

In other embodiments, engine 206—in response to receiving dialogue input—may provide the received dialogue input to a machine learning model for processing dialogue including color encoding and decoding. The machine learning model may then apply decision logic to determine a hierarchal data traversal process for collecting reply to data. In such aspects, engine 206 may associate one or more established rule sets (or models) with a corresponding personalized avatar or agents, according to preferences to avatar display including race and gender and facilitate the deployment and/or implementation of the avatar or agents and rule set (or model) to one or more computing device, service, or subject accounts.

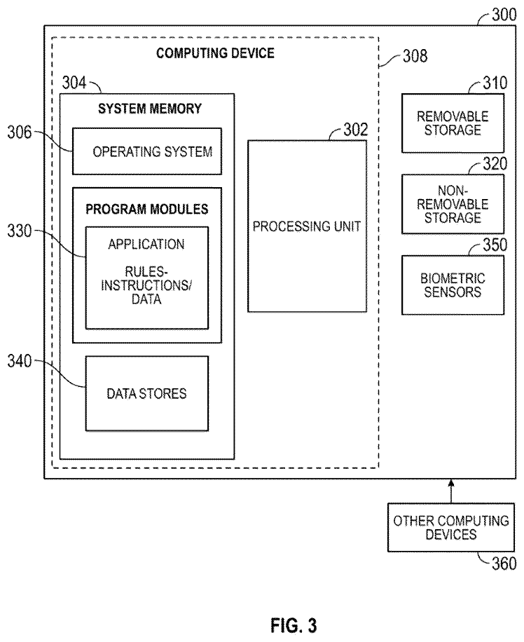

Referring now to FIG. 3, one embodiment of a computing device 300 according to the present invention is illustratively depicted. Device 300 comprises computing device 308, which comprises a processing unit (or device) 302, that receives a request associated with a specific person or entity, input to device 300. Device 308 may further comprise a system memory 304. Depending on the configuration and type of computing device, the system memory 304 may comprise, but is not limited to volatile storage (e.g., random access memory), non-volatile storage (e.g., read-only memory), flash memory, or any combination of thereof.

System memory 304 may comprise an operating system 306 and one or more program modules 330. In certain embodiments, module 330 may be provided for operating a software application 330, such as one or more components supported by the systems described herein. Module 330 may, in other embodiments, comprise one or more sets of personal data (e.g., subject personal data, trauma details, preferences, aspirations, subject profile information, reported disease diagnosis, behavioral data), or instructions for creating an agentic therapy assistant. Operating system 306 may control operation of device 308. Depending upon the embodiment, device 300 may further comprise removable storage device 310, and non-removable storage device 320.

Device 300 may also operatively couple to one or more input device(s)—such as a keyboard, a mouse, a pen, a sound or voice input device, or a touch or swipe input device. Device 300 may further be operatively coupled to other computing device(s) 360—such as a display, speakers, a printer, or a signaling device. Device 300 may further comprise one or more communication connections with other computing devices 360.

Certain embodiments of the present invention may provide unit 302 in the form of unit 200 (depicted in FIG. 2). In certain embodiments, unit 302 may receive a request to generate, train, or modify a chat bot or LU model. Other embodiments of the present invention may provide operations connected with various libraries, other operating systems, or other application programs.

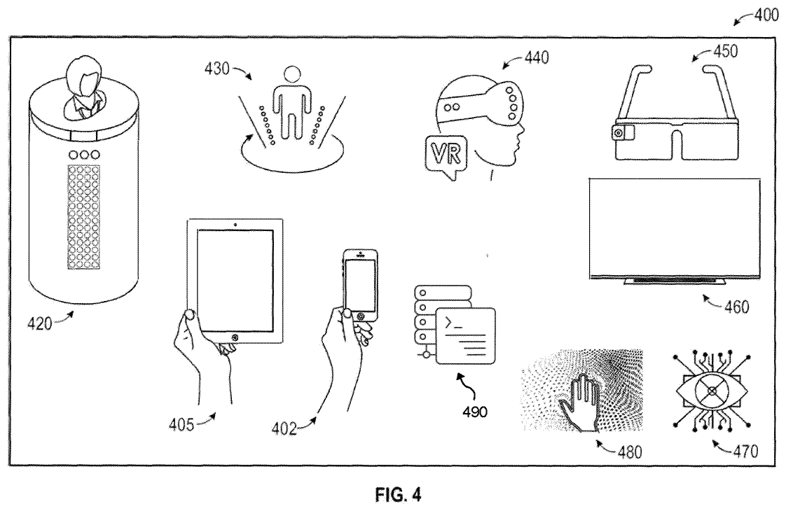

FIG. 4 illustratively depicts a computer system 400 according to the present invention, which may comprise a variety of constituent components for subject output and input. System 400 may comprise various forms of mobile communications (or other interactive) devices. As depicted, one such device may be a mobile phone 402. System 400 may also comprise a tablet computer 405, and an indication device 420. Device 420 may comprise components to provide visual notifications, and/or audible notifications via an audio transceiver. In embodiment depicted, device 420 may comprise a light(s) emitting diode (LED) or other light-emitting system, and an audio speaker with a built-in microphone.

In some embodiments, system 400 may comprise a personal audio/visual interface device 430 (with a 3-D holographic element in certain embodiments), virtual reality or metaverse headset 440, augmented reality or smart glasses 450, television screen 460, eye scanning device 470, a sensory/haptic system 480, or a command line interface 490. Depending upon the embodiment, interface 490 may be provided for input or output for a non-human subject—such as an industrial sensor or system, or a networked device, for example. In other embodiments of the present invention, device 430 comprises an on-board light-emitting system that expresses language in color, ready for translation using biometric sensors, including quantum dots programmed for LU.

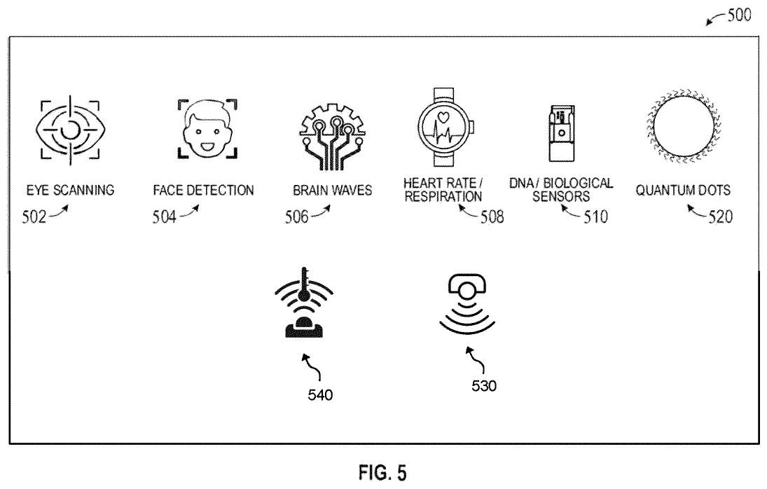

FIG. 5 illustratively depicts various embodiments of an array 500 of sensor devices and/or sensor-enabled devices (collectively referred to as “sensor devices”) according to the present invention. Array 500 may comprise one or more of sensor devices 502-540. According to the present invention, sensor devices are not limited to any number or type of telemetry, biometric, operational, haptic, networked, biological, environmental, or industrial sensors. Depending upon the embodiment, array 500 may comprise an eye scanning sensor 502, such as a retinal scanner. Array 500 may further comprise a face detection sensor 504 comprising a camera element. In certain embodiments, either or both of these sensors may be provided as an application, or application subsystem, on a smart phone. These embodiments may provide data related to eye movements, pupil dilation, blink rates, facial expression, facial tics, or facial affect. In another embodiment, a smart phone application may be provided to measure or collect vital signs of a subject.

Certain other wearable sensor technologies may provide further data concerning the condition of a subject. For example, signal data from device 506 may obtained via some form of wearable device equipped with one or more sensors (e.g., smart glasses, VR headsets, athletic caps, or headbands) or implanted device (e.g., chip, brain-computer interface) that provides measurement or monitoring of brain activity (e.g., brain waves, blood flow). Other wearable devices, such as smart watches, ear buds, or rings, may provide galvanic or other contact-related measurement of bodily function or physiological reactions. Biological sensor 510 may comprise DNA and other biological systems providing measurement or monitoring via biology-gated transistors. Quantum dots 520 may be provided and programmed to accurately communicate data via colors (as a non-verbal language element) and may emit color(s) based upon a stress response. Other embodiments of a sensor device may comprise an industrial sensor 530 or environmental sensor 540 or—in some embodiments—tactile or haptic sensor systems.

The system of the present invention processes and correlates data from the sensor-enabled devices to measure, monitor, or communicate brain waves, brain activity, physiological conditions or impairments, stress response, intent, and emotions—as described throughout this disclosure of the present invention. As those of skill in the art will appreciate, many variations and combinations of sensor devices and sensor data are comprehended by the present invention and fall within its scope.

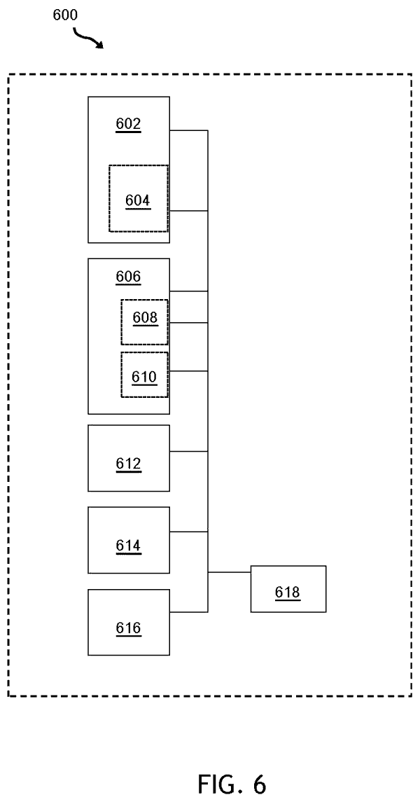

FIG. 6 illustratively depicts an embodiment of a system 600 according to the present invention. System 600 provides a digital system for evaluating one or more responses received from a subject—and comprises a number of components, functions, and operations as described herein. System 600 comprises an evaluation engine 602, that may include a pattern recognition module 604 (which may be optional in certain embodiments).

System 600 may comprise a reference database 606. Depending upon the embodiment, database 606 may comprise a reference (or baseline) profile construct 608 and/or a subject-specific profile construct 610. System 600 further comprises a query engine 612, a digital agent component 614, and one or more sensor devices 616. System 600 may further comprise a contextualization or data unification module 618. All the constituent components of system 600 are operatively and communicatively intercoupled.

Module 618 processes data from a subject's response to a query or guidance—combining, linking, or merging the response data with response characteristic data that accompanies the response. In other words, module 618 generates unified data for any given subject response—data that comprises both the response and response characteristic data. The resulting unified data provides contextualization for responses.

In certain embodiments, module 604 may dynamically identify patterns or trends in data supplied to engine 602. In such embodiments, module 604 may identify whether a subject response meets a certain threshold, falls within a certain range, or is consistent with data retrieved from database 606. Reference database 606 may transfer data to engine 602 from reference profile 608 or subject profile 610.

Engine 612 provides or generates queries for a subject that may be retrieved from a (standalone) database of possible queries or dynamically generated according to certain rules or parameters based upon data retrieved from database 606. Agent 614 provides interaction with a subject in real time using queries or adaptive guidance generated by engine 612. As used in this context, directions may be one form of adaptive guidance presented to a subject. In other embodiments, adaptive guidance may be presented to a subject in the form of advice, assignments, or plans. These are just a few examples of the many embodiments that fall within the scope of the present disclosure.

Sensor devices 616 communicate or record a response characteristic of the subject, while the subject is responding to interaction with agent 614.

Engine 602 evaluates the unified data from module 618 to determine what course of action, if any, is needed as a result. In some instances, engine 602 may trigger an alert, escalation, or message to a user. In other cases, engine 602 may prompt engine 612 and agent construct 614 for follow up or further interaction with a subject, to confirm or clarify a previous response from the subject. In other embodiments, engine 602 may prompt adaptations or modifications to queries or guidance presented by engine 612. In such instances, the process of adapting or modifying queries or guidance may comprise a single iteration or may comprise multiple iterations. In this manner, engine 602 provides adaptive guidance to engine 612 or, more directly, to a subject—responsive to the unified data received by engine 602.

Further aspects of the present invention are illustratively depicted in reference to FIG. 7. As depicted in FIG. 7, an input processing unit 700 is provided for digitizing emotional parameters and latency states for correlation with subject profile data 730. Digitizing emotional parameters and latency states and mapping them to disease conditions 790 may be provided via a combination of operations implemented via components of unit 700.

Based upon receiving data 706 from sensor devices or other functional or biological subsystems, ML engine 708 may access certain subject responses 740, subject profile data 730, and/or stored rules/training data 720. Engine 708 may evaluate such data, and data 706, to determine or project a confidence or genuineness level score for responses 710.

In certain embodiments, a user (therapist) may provide adaptive guidance 702 to a subject—directing the subject to experience a particular feeling for 10-15 seconds (704A) or repeat a latency state at least two times (704B). The subject may comply with the direction, providing responses 740 while sensor devices measure data 706 and engine 708 determines a confidence level score 710. The responses 740 may be evaluated by engine 708 by processing data 730 with rules 720 to generate response score 710. Unit 700 provides a mapping of disease condition 750 to emotional parameters and latency states 780, based upon a frequency of similarities between subject profile data 730 for multiple subjects having similar reported disease condition, emotions, and latency states. The resultant match or correlation data 790 is stored within a database or other suitable data repository.

Communications between a user 702, subject (704A-B), and engine 708 may be provided in any suitable form—such as voice to text conversion—for processing by engine 108.

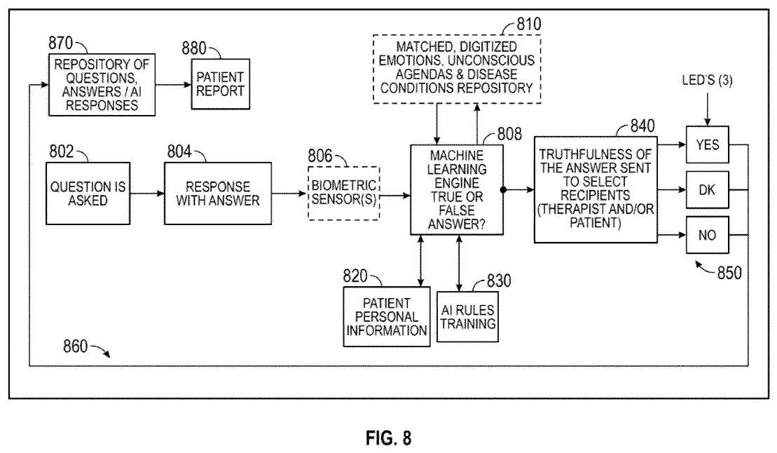

FIG. 8 illustratively depicts an embodiment similar to the one depicted in FIG. 7. Input processing unit 800 is provided such that a therapist 802 asks a queries a subject 804 while sensor devices 806 monitor the subject. Sensor data from the subject is received by a ML engine 808—which processes that data by comparing and contrasting the subject's digitized emotional parameters, latency states, and disease conditions 810 with data from the subject's profile data 820, in accord with stored rules/training data 830. Profile data 820 may comprise the subject's most commonly experienced emotions, latency states, and reported disease conditions that have been medically diagnosed. Based upon the subject's response(s) 850, engine 808 may generate a response score 840.

Score 840 may be stored in a repository 870 that—in certain embodiments—may be accessed during the processing of related data by engine 808. Unit 800 may provide a user report 880 which, depending upon the embodiment, may provide the user data correlating emotional risk factors with underlying disease conditions. The constituent components of unit 800 may operate simultaneously, and be updated in real-time (or otherwise), and may utilize feedback loop(s) to provide training or learning over time and through experience garnered from multitudinous results reports 880.

In various embodiments, engine 808 may provide evaluation of truthful or untruthful responses 804 from a subject in response to queries posed by a user 802. In some embodiments, user 802 may be a therapist or other clinician, while subject 804 is a patient or client. In other embodiments, user 802 may be an evaluator in any field, industry, category, or position—evaluating job performance, hiring success predictability, lie detection, loan applicant quality, or functional performance, for example.

For purposes of illustration and explanation, various application-specific embodiments and implementations of the present invention are now described in further detail.

In some implementations, a modular system may integrate a conversational AI agent with clinical-grade and non-contact biometric sensors, a real-time contextualization engine, and a data structuring module to streamline healthcare workflows.

The conversational AI agent may interact with patients using natural language through various computing interfaces, including text-based chatbots, speech-based voice agents, avatar-based interfaces, or remote human agents operating through the system. This agent may engage with patients at multiple points in their care journey, such as before, during, or after clinical encounters, and may facilitate between-visit communication, longitudinal tracking, and contextualization of health data. To enhance usability, the agent may include a dialogue manager that prompts patients to perform measurements, confirms sensor data, issues adaptive follow-up guidance for incomplete or invalid inputs, and dynamically adjusts session flow based on real-time patient states or measurement results. Multilingual interaction may also be supported, allowing patients to select their preferred language for intake questions, with responses translated into clinical terminology standards, such as Systematized Nomenclature of Medicine Clinical Terms (SNOMED CT), Logical Observation Identifiers Names and Codes (LOINC), or the International Classification of Diseases, Tenth Revision (ICD-10).

In some implementations, the system may integrate clinical-grade and non-contact biometric sensors to collect physiological and neurological data. Clinical-grade sensors may include devices such as blood pressure cuffs, pulse oximeters, thermometers, weight scales, and respiratory rate sensors, while non-contact biometric sensors may utilize cameras and microphones to extract indicators like pulse rate, respiratory effort, vocal biomarkers, or facial micro expressions. Multi-parameter devices may capture multiple vital signs simultaneously, such as blood pressure, heart rate, blood oxygen saturation, respiratory rate, and body temperature, through a single sensor or patch-based configuration.

Additionally, the system may optionally provide non-clinical wearable devices, such as smartwatches or fitness trackers, to provide supplemental data on activity levels, sleep patterns, or heart rate trends. Emerging non-contact sensing technologies, such as remote photoplethysmography for pulse and respiratory data collection using facial skin tone variation or acoustic signal analysis for deriving heart rate variability and stress markers, may also be supported, enabling data collection in scenarios where traditional sensors are unavailable or impractical.

In some implementations, a workflow engine may guide patients through a step-by-step process for using sensor devices or engaging in contact or contactless sensing. This engine may track session state and intake completeness, ensuring that all required data is collected during the intake process.

A real-time evaluation engine may analyze physiological signals, such as blood pressure, oxygen saturation, heart rate, and temperature, to confirm whether measurements are properly obtained and fall within acceptable ranges or confidence intervals. If a measurement is invalid or incomplete, the evaluation engine may prompt the patient—via a conversational agent or chat interface, for example-to retry the measurement with corrective instructions, ensuring that only accurate, high-confidence data is transmitted to downstream clinical systems. The system may also include a data structuring module that formats and transmits validated or inferred health data to clinical systems for provider access, triage decision-making, or patient record integration. This module may support standardized protocols, such as Health Level Seven (HL7) or Fast Healthcare Interoperability Resources (FHIR), and ensure that all data is timestamped, encrypted, and securely transmitted to electronic health record (EHR) systems.

In some implementations, the system may automate the collection and contextualization of vital signs, including blood pressure, pulse rate, blood oxygen saturation, respiratory rate, body temperature, heart rate variability, weight, and height. The conversational AI agent may guide patients through the correct use of medical devices, ensuring procedural accuracy and proper application. The system may support modular integration of advanced bio signal capture tools, such as electrocardiogram (ECG) devices for arrhythmia detection, electroencephalogram (EEG) sensors for cognitive workload assessment, and remote patient monitoring devices for home-based care. Additionally, the system may include a paperless intake module that collects structured clinical data, such as reason for visit, medication lists, allergy reviews, insurance information, and responses to standardized assessments like the Patient Health Questionnaire-9 (PHQ-9). This data may be used to enhance triage workflows, with an intelligent triage engine analyzing patient-collected data, such as symptoms, vitals, and recent complaints, alongside EHR data, such as recent labs, visit history, and diagnosis codes, to assign urgency levels and prioritize care based on acuity.

In some implementations, the system may include an emotional state detection module that interprets patient signals, such as voice tone, biometric variation, or facial expression, to infer affective states like stress levels, affective tone, or cognitive load. These indicators may be incorporated into the patient profile to support triage decisions or behavioral health flagging. The system may also integrate with neurobiological sensing interfaces, such as EEG sensors, to assess cognitive workload, attention levels, or affective states, enabling dynamic adjustments to intake flows or escalation to behavioral health pathways.

For patients identified as experiencing emotional distress or elevated PHQ-9 scores, a behavioral risk follow-up module may schedule follow-up check-ins to assess safety, emotional state, and patient-reported outcomes, with options to escalate to live telehealth sessions or refer to behavioral health support. Furthermore, the system may enable post-visit communication, allowing the conversational AI agent to engage patients in follow-up check-ins, monitor symptom trends, provide medication reminders, and transmit structured updates to provider-facing dashboards or affiliated remote monitoring services.

In some implementations, the system may include context-aware flow control, allowing the conversational AI agent to alter the intake flow in real time based on patient responses or physiological data. For example, the system may skip a mental health screener if critical vitals are abnormal, or reorder questions based on urgency. Real-time sensing logic may also be incorporated to assess whether a patient is actively deteriorating or stabilizing during the intake session, with changes in voice, pacing, and biometric patterns, such as heart rate variability, respiratory rhythm, blood pressure, or oxygenation trends, tracked to flag sessions for escalation or notify supervising personnel. The system may further include an ambient AI documentation module that passively listens during provider-patient encounters to detect structured clinical statements relevant to documentation. This module may generate draft clinical notes, flag incomplete documentation, or surface review prompts in the EHR inbox, streamlining administrative workflows for providers.

In some implementations, the system may integrate with EHR platforms, enabling seamless transmission of structured intake data, vitals, and patient responses. Supported platforms may include outpatient practice systems, cloud-based primary care systems, and large-scale health system interoperability platforms. The system may be designed in accordance with healthcare data privacy and security standards, including encryption of data in transit and at rest, and may ensure that patients retain rights to their data under applicable laws. Deployment may align with partner-specific data ownership and consent requirements, supporting use in various healthcare environments, such as primary care clinics, urgent care centers, telehealth platforms, home-based care settings, behavioral health facilities, senior living communities, and post-acute rehabilitation centers.

To ensure future compatibility, the system may support modular extensions, including emotional signal sensing, neurobiological signal integration, AI-powered triage, and predictive modeling, as well as emerging technologies like non-contact sensing modalities, wearable device inputs, and brain-computer interface devices or sensing implants. These features may position the system as a compliant, adaptable interface for ambient sensing in constrained or non-traditional clinical environments.

Aspects of the present invention may be implemented to support enhanced patient engagement, by enabling dynamic conversational interactions that may adapt to individual health profiles and intake scenarios. The system may improve operational efficiency by automating intake workflows that may reduce manual data entry and procedural redundancies. The modular architecture may facilitate integration with diverse healthcare environments, allowing deployment across remote, in-clinic, or hybrid care settings. The use of real-time contextualization engines may ensure the accuracy and reliability of collected physiological data, which may enhance clinical decision-making and patient safety.

The system of the present invention may support compliance with healthcare data privacy standards by encrypting sensitive information and ensuring secure transmission to clinical systems. The described features may enable scalable solutions for healthcare providers seeking to optimize intake processes while maintaining flexibility for future extensions and emerging technologies.

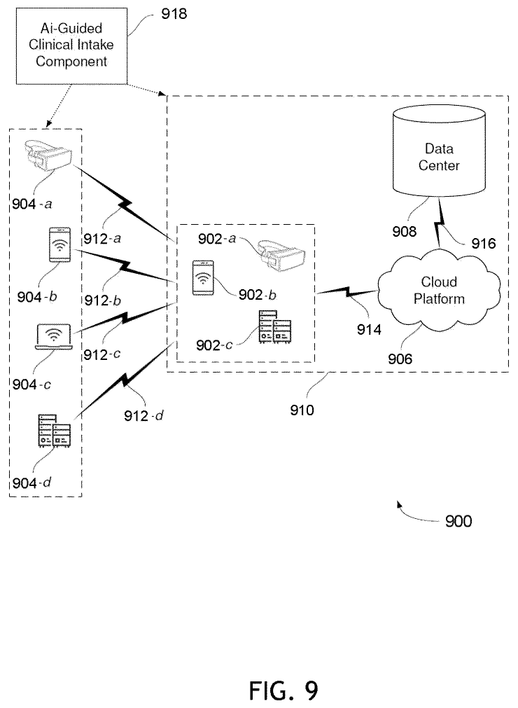

FIG. 9 illustrates an example of a system 900 that supports AI-guided clinical intake with biometric sensors and real-time data contextualization in accordance with various aspects of the present invention. System 900 includes cloud clients 902, user devices 904, a cloud platform 906, and a data center 908. Cloud platform 906 may be an example of a public or private cloud network. A cloud client 902 may access cloud platform 906 over a network connection 914. The network connection 914 may include a wired connection, a wireless connection, or both. The network may implement transfer control protocol and internet protocol (TCP/IP), such as the Internet, or may implement other network protocols.

Cloud client 902 may be an example of a computing device, such as a wearable device (e.g., cloud client 902-a), a smartphone (e.g., cloud client 902-b), or a server (e.g., cloud client 902-c). In other examples, a cloud client 902 may be a desktop or laptop computer, a tablet, a sensor, or another computing device or system capable of generating, analyzing, transmitting, or receiving communications. In some examples, a cloud client 902 may be part of a business, an enterprise, a non-profit, a startup, or any other organization type.

Cloud client 902 may facilitate communication between the data center 908 and one or multiple user devices 904 to implement an online environment. The network connection 912 may include communications, opportunities, purchases, sales, or any other interaction between a cloud client 902 and a user device 904. Network connection 912 may include a wired connection, a wireless connection, or both. A cloud client 902 may access cloud platform 906 to store, manage, and process the data communicated via one or more network connections 912. In some cases, the cloud client 902 may have an associated security or permission level. A cloud client 902 may have access to certain applications, data, and database information within cloud platform 906 based on the associated security or permission level and may not have access to others.

The user device 104 may include an AI-guided clinical intake component 118. The user device 104 may interact with the cloud client 102 over network connection 112. The network may implement transfer control protocol and internet protocol (TCP/IP), such as the Internet, or may implement other network protocols. The network connection 112 may facilitate transport of data via email, web, text messages, mail, or any other appropriate form of electronic interaction (e.g., network connections 112-a, 112-b, 112-c, and 112-d) via a computer network. In an example, the user device 104 may be computing device such as a wearable device 104-a, a smartphone 104-b, a laptop 104-c, or a server 104-d. In other cases, the user device 104 may be another computing system. In some cases, the user device 104 may be operated by a user or group of users. The user or group of users may be a customer, associated with a business, a manufacturer, or any other appropriate organization.

Cloud platform 906 may offer an on-demand database service to the cloud client 902. In some cases, cloud platform 906 may be an example of a multi-tenant database system. In this case, cloud platform 906 may serve multiple cloud clients 902 with a single instance of software. However, other types of systems may be implemented, including—but not limited to—client-server systems, mobile device systems, and mobile network systems. In some cases, cloud platform 906 may support an online application. This may include support for sales between buyers and sellers operating user devices 904, service, marketing of products posted by buyers, community interactions between buyers and sellers, analytics, such as user-interaction metrics, applications (e.g., computer vision and machine learning), and the Internet of Things (IoT).

Cloud platform 906 may receive data associated with generation of an online environment from the cloud client 902 over network connection 914 and may store and analyze the data. In some cases, cloud platform 906 may receive data directly from a user device 904 and the cloud client 902. In some cases, the cloud client 902 may develop applications to run on cloud platform 906. Cloud platform 906 may be implemented using remote servers. In some cases, the remote servers may be located at one or more data centers 908.

Data center 908 may include multiple servers. The multiple servers may be used for data storage, management, and processing. Data center 908 may receive data from cloud platform 906 via connection 916, or directly from the cloud client 902 or via network connection 912 between a user device 904 and the cloud client 902. The connection 916 may include a wired connection, a wireless connection, or both. Data center 908 may utilize multiple redundancies for security purposes. In some cases, the data stored at data center 908 may be backed up by copies of the data at a different data center (not pictured).

Server system 910 may include cloud clients 902, a cloud platform 906, an AI-guided clinical intake component 918, and a data center 908 that may coordinate with cloud platform 906 and data center 908 to implement an online environment. In some cases, data processing may occur at any of the components of server system 910, or at a combination of these components. Thus, the AI-guided clinical intake component 918 may be included in the user device 904, server system 910, or in part or in whole in both. In some cases, servers may perform the data processing. The servers may be a cloud client 902 or located at data center 908.

Some or all of the functionality attributed to the AI-guided clinical intake component 918 may be embodied or performed by one or more user devices 904, one or more components of server system 910 (e.g., cloud clients 902, a cloud platform 906, and/or a data center 908), and/or other components of system 900. The AI-guided clinical intake component 918 may receive signals and inputs from user device 904 directly. via cloud clients 902, and/or via cloud platform 906 or data center 916.

As described herein, the AI-guided clinical intake component 918 may facilitate the collection and contextualization of patient health data by interfacing with biometric sensing devices integrated into user devices 910 or connected via network connections 912. The component may analyze physiological signals received from clinical-grade sensors or non-contact biometric sensors to identify patterns or anomalies indicative of specific health states. These patterns or anomalies may be validated in real time by comparing the data against predefined acceptable ranges or confidence intervals. The validated data may then be structured into a standardized format compatible with electronic health record systems and ranked based on clinical risk scoring. The ranked data may be transmitted to cloud clients 902 or cloud platform 906 for integration into patient records or triage decision-making processes, enabling prioritization of care based on acuity.

It should be appreciated by a person skilled in the art that one or more aspects of the disclosure may be implemented in a system 900 to solve other problems additionally or alternatively than those described above.

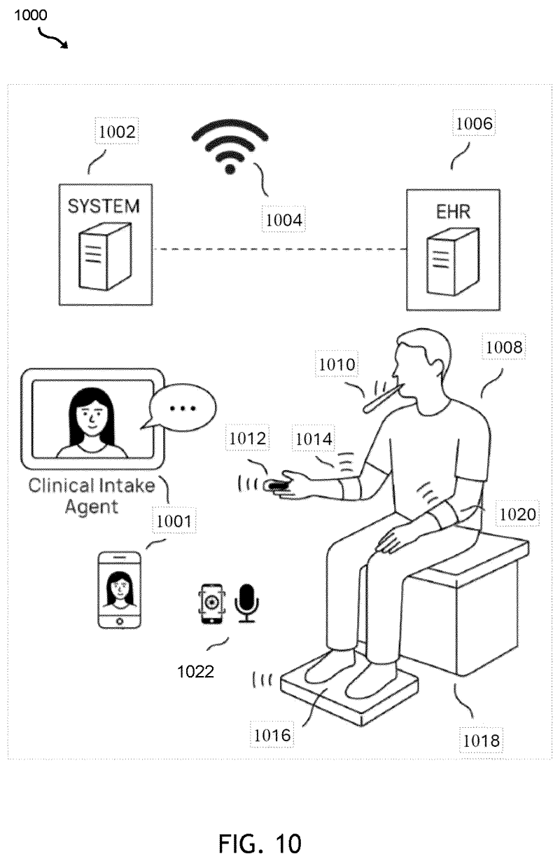

FIG. 10 shows clinical intake system 1000 which supports techniques for AI-guided clinical intake with biometric sensors and real-time data contextualization in accordance with various aspects of the present disclosure. As depicted in FIG. 10, the clinical intake system 1000 may include one or more of a clinical intake agent interface 1001, a clinical intake agent system server 1002, a wireless signal 1004, an electronic health record system (EHR) 1006, a patient 1008, a wireless thermometer 1010, a pulse oximeter sensor 1012, a blood pressure cuff 1014, an electronic scale 1016, a bench 1018, a wearable sensor device 1020, a non-contact biometric sensor 1022, and/or other components.

The clinical intake agent interface 1001 may include a digital platform configured to interact with patients through natural language prompts and visual guidance. The clinical intake agent interface 1001 may include a touchscreen display or voice-activated system that may allow patients to engage in guided intake sessions. The interface may be configured to present intake questions in a patient-selected language and may translate responses into a clinical provider language. In some implementations, the clinical intake agent interface 1001 may be deployed in healthcare settings such as exam rooms, telehealth platforms, emergency care facilities, or home-care environments.

The clinical intake agent system server 1002 may represent a backend infrastructure designed to manage conversational logic, contextualization of sensor data with subject-reported behaviors or outcomes, and workflow validation or coordination. The clinical intake agent server 1002 may include a secure, network-connected infrastructure that may host the conversational AI software and manage real-time data flow between devices and electronic health record systems. The server may be configured to validate vital sign measurements or derived signals to confirm they fall within a physiologically acceptable range or predetermined confidence interval. In some implementations, the clinical intake agent system server 1002 may transmit structured data compatible with HL7 or FHIR protocols to clinical systems or other structured data protocols.

The wireless signal 1004 may provide a communication pathway for transmitting data between biometric sensors and the clinical intake agent interface. The wireless signal 1004 may include protocols such as Bluetooth or Wi-Fi that may enable real-time data exchange between connected devices and the clinical intake agent system. The wireless signal 1004 may support communication between clinical-grade sensors such as a blood pressure cuff 1014 or a pulse oximeter sensor 1012 and the clinical intake agent interface 1001. In some implementations, the wireless signal 1004 may be used to transmit data from non-contact biometric sensors 1022 to the clinical intake agent system server 1002.

The electronic health record system (EHR) 1006 may include a structured database configured to receive and store validated patient data transmitted from the clinical intake agent system. The electronic health record system (EHR) 1006 may include platforms such as Epic, Oracle, or MyChart that may integrate patient intake data for provider access and triage decision-making. The EHR system may normalize patient responses to clinical terminology standards such as SNOMED CT, LOINC, or ICD-10. In some implementations, the electronic health record system (EHR) 1006 may support live or asynchronous deployment in telehealth or remote monitoring environments.

The patient 1008 may represent an individual engaging with the clinical intake agent interface to complete intake tasks and biometric measurements. The patient 1008 may interact with the conversational agent to perform vital sign measurements or respond to standardized clinical assessments. The patient 1008 may use clinical-grade devices such as a wireless thermometer 1010 or a wearable sensor device 1020 during the intake session. In some implementations, the patient 1008 may engage in post-visit communication with the clinical intake agent interface 1001 to monitor symptom trends or receive medication reminders.

The wireless thermometer 1010 may include a clinical-grade device capable of measuring body temperature and transmitting the data wirelessly to the clinical intake agent system. The wireless thermometer 1010 may be positioned in the patient's mouth or on the skin to collect core body temperature readings. The wireless thermometer 1010 may transmit validated temperature data to the clinical intake agent system server 1002 for integration into the electronic health record system (EHR) 1006. In some implementations, the wireless thermometer 1010 may be used in conjunction with other clinical-grade devices such as a pulse oximeter sensor 1012 or a blood pressure cuff 1014.

The pulse oximeter sensor 1012 may provide real-time measurements of blood oxygen saturation and heart rate through a wireless connection to the clinical intake agent interface. The pulse oximeter sensor 1012 may be placed on the patient's finger to collect SpO2 and heart rate data. The pulse oximeter sensor 1012 may transmit validated physiological data to the clinical intake agent system server 1002 for provider access or patient record integration. In some implementations, the pulse oximeter sensor 1012 may be used alongside a wearable sensor device 1020 to capture additional vital signs such as respiratory rate or heart rate variability.

The blood pressure cuff 1014 may include a wireless device capable of capturing systolic and diastolic blood pressure readings and transmitting the data to the clinical intake agent system. The blood pressure cuff 1014 may be applied to the patient's upper arm to measure blood pressure during the intake session. The blood pressure cuff 1014 may transmit validated readings to the clinical intake agent server 1002 for real-time analysis and structured data formatting. In some implementations, the blood pressure cuff 1014 may be part of a multi-parameter sensor device 1020 capable of capturing multiple vital signs simultaneously.

The electronic scale 1016 may represent a wireless device configured to measure patient weight and transmit the data to the clinical intake agent interface. The electronic scale 1016 may be positioned on the floor to allow the patient to step on and measure their weight. The electronic scale 1016 may transmit validated weight data to the clinical intake agent system server 1002 for integration into the electronic health record system (EHR) 1006. In some implementations, the electronic scale 1016 may be used in conjunction with a bench 1018 to support the patient during the intake session.

The bench 1018 may provide physical support for the patient during the intake session without being part of the digital system. The bench 1018 may be positioned in the clinical environment to allow the patient to remain seated while interacting with the clinical intake agent interface 100. The bench 1018 may be used alongside clinical-grade devices such as a wireless thermometer 1010 or a blood pressure cuff 1014 during the intake session. In some implementations, the bench 1018 may be placed in healthcare settings such as exam rooms or urgent care centers.

The wearable sensor device 1020 may include a multi-parameter device capable of capturing vital signs such as heart rate variability and respiratory rate from a single anatomical location. The wearable sensor device 1020 may be positioned on the patient's upper arm or wrist to collect physiological data during the intake session. The wearable sensor device 1020 may transmit validated data to the clinical intake agent system server 1002 for provider access or triage decision-making. In some implementations, the wearable sensor device 1020 may be used alongside non-contact biometric sensors 1022 to capture additional indicators such as pulse rate or vocal biomarkers.

The non-contact biometric sensor 1022 may represent a camera or microphone capable of extracting physiological or neurological indicators such as pulse rate or vocal biomarkers. The non-contact biometric sensor 1022 may use remote photoplethysmography (rPPG) to estimate pulse rate or respiratory effort from facial skin tone variation. The non-contact biometric sensor 1022 may transmit validated data to the clinical intake agent system server 1002 for integration into the electronic health record system (EHR) 1006. In some implementations, the non-contact biometric sensor 1022 may be used in telehealth platforms or home-care environments.

In some implementations, the clinical intake agent interface 1001 may interact with the patient 1008 through a display screen, guiding the patient in real-time as they engage with various biometric sensing devices. The clinical intake agent system server 1002 may communicate wirelessly via the wireless signal 1004 to both the electronic health record system (EHR) 1006 and the connected biometric sensors. The patient 1008 may use the wireless thermometer 1010 to measure body temperature, while the pulse oximeter sensor 1012 may capture blood oxygen saturation and pulse rate data.

In some implementations, the blood pressure cuff 1014 may be applied to the patient 1008 to measure blood pressure, and the electronic scale 1016 may detect weight data as the patient stands on it. The bench 1018 may serve as a seating area for the patient 1008 during the intake process, allowing them to interact with the wearable sensor device 1020, which may transmit contextual physiological data such as activity levels or heart rate trends. The non-contact biometric sensor 1022 may capture additional signals, such as vocal biomarkers or respiratory effort, through microphones or cameras, transmitting this data wirelessly to the clinical intake agent system server 1002 for real-time validation and data structuring.

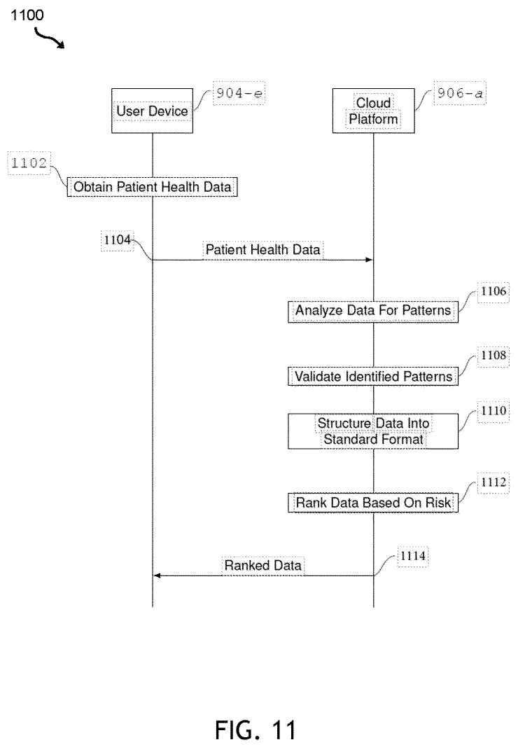

FIG. 11 illustrates an example of a process flow 1100 that supports AI-guided clinical intake with biometric sensors and real-time data contextualization in accordance with aspects of the present disclosure. In some examples, the process flow 1100 may implement aspects of the system 900. For example, the process flow 1100 may include a user device 904-e and a cloud platform 906-a, which may be examples of corresponding devices described herein. In some implementations, a user device 904-e collects patient health data via biometric sensors and transmits the data to a cloud platform 906-a, which analyzes, validates, structures, ranks, and integrates the data into clinical systems for electronic health records or triage decision-making.

At 1102, the user device 904-e may obtain patient health data via biometric sensing devices, including physiological signals collected by clinical-grade sensors or non-contact biometric sensors. For example, the user device 904-e may receive data from a wireless pulse oximeter configured to measure blood oxygen saturation and heart rate. In some implementations, the user device 904-e may interact with a camera-based non-contact sensor to determine pulse rate or respiratory effort through remote photoplethysmography. In other implementations, the user device 904-e may obtain vocal biomarkers from a microphone to determine stress levels or emotional state.

At 1104, the user device 904-e may transmit the obtained patient health data to the cloud platform 906-a for further processing. For example, the user device 904-e may send structured data from a wireless thermometer measuring body temperature to the cloud platform 906-a. In some implementations, the user device 904-e may transmit data from a multi-parameter sensor device capturing blood pressure, heart rate, and respiratory rate to the cloud platform 906-a. In other implementations, the user device 904-e may relay data from a camera-based non-contact biometric sensor extracting pulse rate and skin tone variations to the cloud platform 906-a.

At 1106, the cloud platform 906-a may analyze the transmitted data to identify patterns or anomalies indicative of physiological or neurological states. For example, the cloud platform 906-a may determine variations in heart rate trends from data received from a wireless pulse oximeter. In some implementations, the cloud platform 906-a may identify irregular respiratory rhythms by analyzing data transmitted from a camera-based non-contact biometric sensor. In other implementations, the cloud platform 906-a may detect stress indicators by interpreting tonal variations and pauses in vocal biomarkers transmitted from a microphone.

At 1108, the cloud platform 906-a may validate the identified patterns or anomalies in real time by comparing the data against predefined acceptable ranges or confidence intervals. For example, the cloud platform 906-a may determine whether heart rate variability data falls within expected thresholds by analyzing signals received from a wearable sensor. In some implementations, the cloud platform 906-a may assess respiratory rate data transmitted from a multi-parameter sensor device to confirm its alignment with acceptable physiological ranges. In other implementations, the cloud platform 906-a may evaluate tonal variations in vocal biomarkers to determine whether stress indicators meet predefined confidence metrics.

At 1110, the cloud platform 906-a may structure the validated data into a standardized format compatible with electronic health record systems. For example, the cloud platform 906-a may format the validated data into HL7 or FHIR protocols to ensure compatibility with electronic health record systems. In some implementations, the cloud platform 906-a may organize the data into structured templates that align with clinical terminology standards such as SNOMED CT, LOINC, or ICD-10. In other implementations, the cloud platform 906-a may segment the data into discrete fields for integration into specific modules within electronic health record systems, such as patient history, vitals tracking, or medication reconciliation.

At 1112, the cloud platform 906-a may rank the structured data based on clinical risk scoring in response to detected anomalies or predefined thresholds. For example, the cloud platform 906-a may assign priority levels to patient cases based on combined inputs such as abnormal vital signs, recent symptom reports, or flagged medication interactions. In some implementations, the cloud platform 906-a may determine urgency scores by analyzing historical trends in patient health data alongside real-time measurements, such as elevated blood pressure or irregular heart rate patterns. In other implementations, the cloud platform 906-a may group patient cases into risk categories, such as high, medium, or low, based on the severity of detected anomalies and their alignment with predefined clinical thresholds.

At 1114, the cloud platform 906-a may transmit the ranked data to clinical systems for integration into patient records or triage decision-making. For example, the cloud platform 906-a may send ranked data to an electronic health record system configured to display patient priority levels for clinician review. In some implementations, the cloud platform 906-a may transmit ranked data to a triage dashboard that organizes patient cases based on urgency scores derived from combined inputs such as abnormal vital signs and recent symptom reports. In other implementations, the cloud platform 906-a may relay ranked data to a remote monitoring platform that categorizes patient cases into risk groups for further evaluation by healthcare personnel.

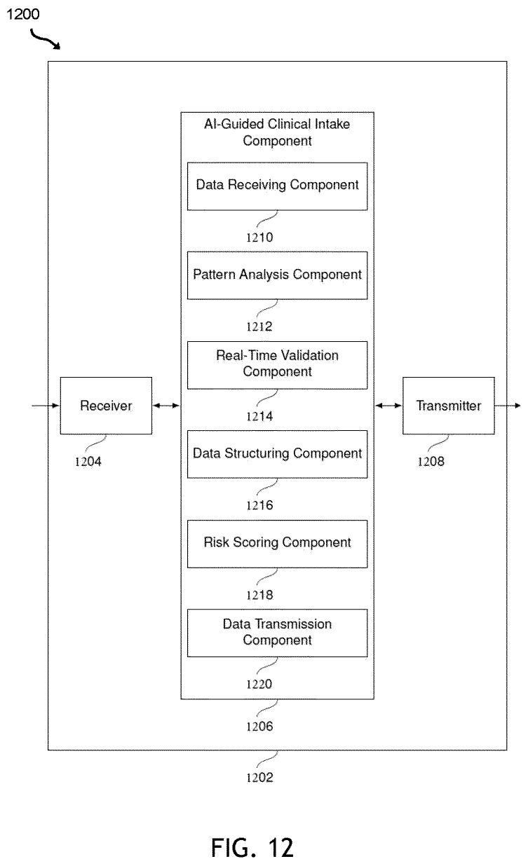

FIG. 12 shows a block diagram 1200 of an apparatus 1202 that supports AI-guided clinical intake with biometric sensors and real-time data validation in accordance with various aspects of the present disclosure. The apparatus 1202 may include an input module 1204, AI-guided clinical intake component 1206, and an output module 1208. The apparatus 1202 may also include a processor. Each of these components may be in communication with one another (e.g., via one or more buses). In some cases, the apparatus 1202 may be an example of a user terminal, a database server, or a system containing multiple computing devices.

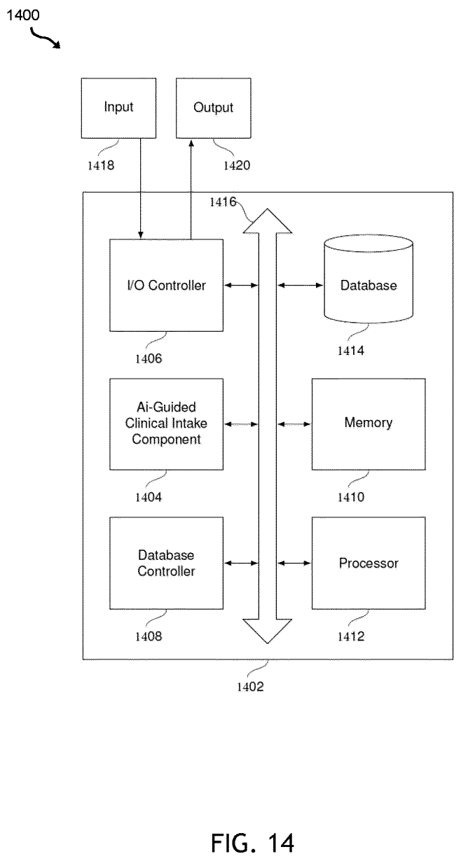

The input module 1204 may manage input signals for the apparatus 1202. For example, the input module 1204 may identify input signals based on an interaction with a modem, a keyboard, a mouse, a touchscreen, or a similar device. These input signals may be associated with user input or processing at other components or devices. In some cases, the input module 1204 may utilize an operating system such as iOS®, ANDROID®, MS-DOS®, MS-WINDOWS®, OS/2®, UNIX®, LINUX®, or another known operating system to manage input signals. The input module 1204 may send aspects of these input signals to other components of the apparatus 1202 for processing. In some cases, the input module 1204 may be a component of an input/output (I/O) controller 606 as described with reference to FIG. 14.

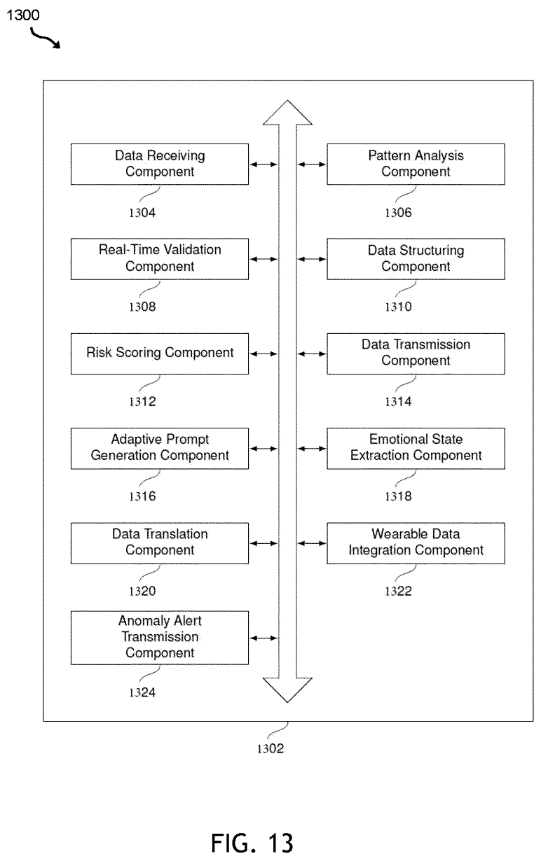

The AI-guided clinical intake component 1206 may include one or more of a data receiving component 1210, a pattern analysis component 1212, a real-time validation component 1214, a data structuring component 1216, a risk scoring component 1218, a data transmission component 1220, and/or other components. The AI-guided clinical intake component 1206 may be an example of aspects of the AI-guided clinical intake component 1302 or 1404 described with reference to FIGS. 13 and 14.

The data receiving component 1210 may be configured as or otherwise support a means for receiving patient health data from biometric sensing devices, the data including physiological signals collected by clinical-grade sensors or non-contact biometric sensors. The pattern analysis component 1212 may be configured as or otherwise support a means for analyzing the received data to identify patterns or anomalies indicative of physiological or neurological states. The real-time validation component 1214 may be configured as or otherwise support a means for validating the identified patterns or anomalies in real time by comparing the data against predefined acceptable ranges, baselines, or confidence intervals. The data structuring component 1216 may be configured as or otherwise support a means for structuring the validated data into a standardized format compatible with electronic health record systems. The risk scoring component 1218 may be configured as or otherwise support a means for ranking the structured data based on clinical risk scoring in response to detected anomalies or predefined thresholds. The data transmission component 1220 may be configured as or otherwise support a means for transmitting the ranked data to clinical systems for integration into patient records or triage decision-making.

Data receiving component 1210, in the clinical context (for example) provides automated ingestion of diverse data sources, such as but not limited to, EHR extracts, structured lab data, connected device feeds, patient demographics, and free-text or voice input. As a standalone module, data receiving component 1210 can leverage any of a variety of AI paradigms either alone or in combination. These AI paradigms include but are not limited to Rule-Based AI, which aids in rigid compliance (e.g., format verification, basic field presence checks); Supervised Machine Learning (ML), which aids in intake error detection, source reconciliation, record linkage, and outlier flagging, especially as datasets become annotated with intake issues; Unsupervised Learning, which aids in anomaly detection in data streams lacking labeled errors or concept drift; Natural Language Processing (NLP), which aids in parsing semi-structured or unstructured fields, e.g., voice-transcribed intake interviews, insurance scans, and OCR-processed documents. In some implementations data receiving component 1210 can support patients in their native language, auto translating and normalizing terminology for downstream modules. Further, data receiving component 1210 may be configured to automate mapping of all major structured and unstructured data sources, leveraging AI to suggest preprocessing steps and impute missing values with k-Nearest Neighbor (k-NN) or regression algorithms. Further still, data receiving component 1210 may be configured to automate web, mobile, and voice-based patient self-intake, using AI to structure consent, verify insurance, and pre-check history, with FHIR/HL7-ready output for EHR integration.

In some implementations, pattern analysis component 1212 may be configured to translate crude intake data into actionable clinical features using any one of or a combination of AI paradigms including, but not limited to, Unsupervised Learning (UL), such as K-means, hierarchical clustering or Density-Based Spatial Clustering of Applications with Noise (DBSCAN) for unearthing patient groups with similar symptoms or histories, which may be a key in remote triage, stratifying for downstream risk scoring or tailored questioning; Deep Learning, which may be configured as an autoencoder, a transformer, and a document embedder (e.g., Doc2Vec, Med-BERT), for high-dimensional EHR/time-series extraction, and pattern recognition in combined text and structured data; and Dimensionality Reduction, such as Principal Component Analysis (PCA), or t-Distributed Stochastic Neighbor Embedding (t-SNE) to visualize and compress features for meaningful decision boundaries and regulatory transparency.

In some particular implementations, it may be necessary to embed data streams which transform sequences of intake events (e.g., meds, labs) into dense patient vectors, which are then clustered for sub-population analytics, early risk detection, or personalized triage protocols. In some particular implementations, it may be necessary to support multimodal pattern analysis, for example merging sensor data, text, and images, with deep learning-powered feature extraction for richer clinical insight and longitudinal memory for a comprehensive, simultaneous analysis or summary.

In some implementations real-time validation component 1214 is configured to ensure data integrity and safety at the point of intake. This function may be accomplished using any one of or a combination of AI paradigms including, but not limited to, Rule-Based Systems which aid in providing deterministic, regulatory-mandated checks e.g., flagging missing mandatory fields, enforcing value ranges/coding standards, or PHI redaction; Reinforcement Learning (RL), which may aid in adaptive contextualization, for example learning which data points, instruments, or patient-reported outcomes require escalation or human confirmation by maximizing the reward (e.g., data accuracy, workflow speed, risk mitigation); and Hybrid AI, which aids in combining static rules (for regulatory compliance) with RL-driven dynamic escalation pathways, explainable anomaly detection, and contextual alerting.

As an example, if a patient input triggers a red flag (e.g., severe chest pain), the agentic real-time validation component 1214 can autonomously escalate to human review while logging the episode for continuous RL policy tuning. In some implementations, RL may be used to learn how best to triage ambiguous symptoms, update question flows, and minimize false escalations over time.

Data structuring component 1216 may be configured to take the raw and variably formatted intake data, especially free-text, and systematically encode it into computable, analyzable, and interoperable forms. This function may be accomplished using any one of or a combination of AI paradigms including, but not limited to, Nonlinear Programming (NLP) which aids to extract medications, symptoms, labs, and temporal relationships from raw text, mapping them into SNOMED, ICD-10 or FHIR resources; Generative AI/Large Language Models (LLMs), which aids to turn loosely structured interviews, voice transcripts, and multilingual input into standardized summaries, structured SOAP notes, and clinical orders ready for provider review; and Autoencoders which aid to compress high-dimensional notes for anomaly detection, trend analysis, and semantic deduplication.

### Risk Scoring Component 1218 May Be Configured to Quantify Patient Acuity,

clinical deterioration likelihood, readmission risk, and other adverse events to prioritize resource allocation and escalate urgent cases. This function may be accomplished using any one of or a combination of AI paradigms including, but not limited to, Supervised Learning, such as gradient boosting (e.g. XGBoost), random forests, logistic and Cox regression, and deep neural networks which blend static intake with streaming data for time-dependent risk prediction; Hybrid and Explainable AI such as ensemble classifiers (voting classifiers), Shapley Additive Explanations (SHAP) for feature explainability, and symbolic/logical constraints for regulatory transparency and fairness; and Rule-Driven and Expert Systems rule overlays which ensure clinically interpretable mappings and guardrails as required by FDA, EMA, and Joint Commission standards.

Data transmission component 1220 may be configured for reliable and secure data transmission. It is vital for ensuring that validated, structured, and risk-prioritized intake flows into EHRs, registries, and analytic systems. This function may be accomplished using any one of or a combination of AI paradigms including, but not limited to, Federated Learning/Privacy-Preserving AI which is sensitive to intake data that can stay on-site or edge devices, transmitting only model updates, not raw PHI, for aggregate model retraining or shared risk calibration across institutions; Secure Pipelines, which may be used for end-to-end encryption, secure enclaves, and API gateways (FHIR/HL7) to exchange patient data in real time while ensuring compliance with HIPAA, GDPR, and ISO27001 standards; and Blockchain and Differential Privacy which may be used for auditability and tamper-proof logs, supporting advanced value-based care, trial data management, and multi-site research collaborations.

Module 1208 may manage output signals for the apparatus 1202. For example, the output module 1208 may receive signals from other components of the apparatus 1202, such as the AI-guided clinical intake component 1206, and may transmit these signals to other components or devices. In some specific examples, the output module 1208 may transmit output signals for display in a user interface, for storage in a database or data store, for further processing at a server or server cluster, or for any other processes at any number of devices or systems. In some cases, the output module 1208 may be a component of an I/O controller 1406 as described with reference to FIG. 14.

FIG. 13 shows a block diagram 1300 of an AI-guided clinical intake component 1302 that supports AI-guided clinical intake with biometric sensors and real-time data contextualization in accordance with various aspects of the present disclosure. The AI-guided clinical intake component 1302 may be an example of aspects of an AI-guided clinical intake component 1206, an AI-guided clinical intake component 604, or both, as described herein. The AI-guided clinical intake component 1302, or various components thereof, may be an example of means for performing various aspects of AI-guided clinical intake with biometric sensors and real-time data contextualization as described herein. For example, the AI-guided clinical intake component 1302 may include one or more of a data receiving component 1304, a pattern analysis component 1306, a real-time contextualization component 1308, a data structuring component 1310, a risk scoring component 1312, a data transmission component 1314, an adaptive prompt generation component 1316, an emotional state extraction component 1318, a data translation component 1320, a wearable data integration component 1322, an anomaly alert transmission component 1324, and/or other components. Each of these components may communicate, directly or indirectly, with one another (e.g., via one or more buses).

The data receiving component 1304 may be configured as or otherwise support a means for receiving patient health data from biometric sensing devices, the data may include physiological signals collected by clinical-grade sensors or non-contact biometric sensors. In some implementations, the data receiving component 1304 may support wireless communication protocols such as Bluetooth or Wi-Fi to receive data from connected devices. The data receiving component 1304 may include compatibility with multi-parameter sensor devices capable of transmitting multiple physiological signals simultaneously, such as blood pressure, heart rate, and respiratory rate.

The data receiving component 1304 may be configured as or otherwise support a means for receiving patient health data from biometric sensing devices, the data may include physiological signals collected by clinical-grade sensors or non-contact biometric sensors. In some implementations, the data receiving component 1304 may support integration with camera-based sensors that determine pulse rate or respiratory effort through remote photoplethysmography. The data receiving component 1304 may include functionality to interpret vocal biomarkers transmitted from microphone-based sensors to determine tonal variations or speech cadence.

The pattern analysis component 1306 may be configured as or otherwise support a means for analyzing the received data to identify patterns or anomalies indicative of physiological or neurological states. In some implementations, the pattern analysis component 1306 may determine trends in heart rate variability to infer stress levels or relaxation states. The pattern analysis component 1306 may interpret fluctuations in respiratory rate to identify irregular breathing patterns that may suggest potential respiratory distress.

In some implementations, the pattern analysis component 1306 may analyze facial micro expressions captured by camera-based sensors to determine emotional states such as anxiety or calmness. The pattern analysis component 1306 may evaluate tonal variations in vocal biomarkers to determine shifts in mood or cognitive workload. The pattern analysis component 1306 may assess skin tone changes detected through remote photoplethysmography to determine circulatory or oxygenation anomalies.

The real-time validation component 1308 may be configured as or otherwise support a means for validating the identified patterns or anomalies in real time by comparing the data against predefined acceptable ranges or confidence intervals. In some implementations, the real-time validation component 1308 may determine whether blood pressure readings fall within clinically accepted systolic and diastolic thresholds. In some implementations, the real-time validation component 1308 may assess heart rate data to determine if the values align with expected ranges for resting or active states.

In some implementations, the real-time validation component 1308 may evaluate respiratory rate measurements to determine if the values correspond to normal breathing patterns under specific conditions. In some implementations, the real-time validation component 1308 may validate body temperature readings by comparing them to predefined thresholds for febrile or hypothermic states. In some implementations, the real-time validation component 1308 may determine the accuracy of oxygen saturation levels by cross-referencing the data with acceptable SpO2 ranges for healthy individuals.

The data structuring component 1310 may be configured as or otherwise support a means for structuring the validated data into a standardized format compatible with electronic health record systems. In some implementations, the data structuring component 1310 may format the validated data into HL7 or FHIR protocols to ensure compatibility with electronic health record systems. In some implementations, the data structuring component 1310 may organize the data into discrete fields such as patient ID, timestamp, and measurement type to align with clinical data standards. In some implementations, the data structuring component 1310 may convert the validated data into a format that supports integration with platforms such as Epic, Oracle Health, or MyChart.

The risk scoring component 1312 may be configured as or otherwise support a means for ranking the structured data based on clinical risk scoring in response to detected anomalies or predefined thresholds. In some implementations, the risk scoring component 1312 may determine priority levels for patient evaluation based on combined inputs such as vital sign deviations and symptom severity. In some implementations, the risk scoring component 1312 may assign urgency scores to patients exhibiting elevated heart rates or abnormal oxygen saturation levels detected during intake. In some implementations, the risk scoring component 1312 may categorize patients into risk tiers based on patterns identified in longitudinal health data, such as recurring respiratory irregularities or fluctuating blood pressure trends.

The data transmission component 1314 may be configured as or otherwise support a means for transmitting the ranked data to clinical systems for integration into patient records or triage decision-making. In some implementations, the data transmission component 1314 may transmit ranked data to electronic health record systems such as Epic or Oracle Health through HL7 or FHIR protocols. In some implementations, the data transmission component 1314 may transmit ranked data to clinical dashboards that display patient prioritization based on risk scores. In some implementations, the data transmission component 1314 may transmit ranked data to remote monitoring platforms that track longitudinal health trends for patients outside clinical settings.

In some examples, the adaptive prompt generation component 1316 may be configured as or otherwise support a means for generating adaptive prompts to guide a patient through corrective actions in response to invalid physiological signals detected during real-time validation. In some implementations, the adaptive prompt generation component 1316 may generate prompts that instruct the patient to reposition a blood pressure cuff to ensure proper alignment with the brachial artery. In some implementations, the adaptive prompt generation component 1316 may generate prompts that suggest the patient remain still and avoid talking during a pulse oximeter reading to reduce signal interference.

In some implementations, the adaptive prompt generation component 1316 may generate prompts that recommend the patient adjust the placement of a thermometer to ensure it is correctly positioned under the tongue. In some implementations, the adaptive prompt generation component 1316 may generate prompts that guide the patient to check the battery level of a connected sensor device if signal transmission issues are detected. In some implementations, the adaptive prompt generation component 1316 may generate prompts that instruct the patient to retry a respiratory rate measurement by taking slow, deep breaths to stabilize the reading.

In some examples, the emotional state extraction component 1318 may be configured as or otherwise support a means for extracting emotional state indicators from voice tone, facial expressions, or biometric variations to supplement the ranking of structured data based on clinical risk scoring. In some implementations, the emotional state extraction component 1318 may determine stress levels by analyzing variations in pitch and cadence within the patient's voice tone during intake interactions. In some implementations, the emotional state extraction component 1318 may interpret facial expressions captured by camera-based sensors to identify indicators such as furrowed brows or tightened lips that may suggest anxiety or discomfort. In some implementations, the emotional state extraction component 1318 may assess biometric variations such as heart rate fluctuations or skin conductance changes to infer emotional states such as calmness or agitation.

In some examples, the data translation component 1320 may be configured as or otherwise support a means for translating patient health data into multiple languages and may normalize the translated data to clinical terminology standards compatible with electronic health record systems. In some implementations, the data translation component 1320 may determine the appropriate language for translation based on patient preferences stored in the electronic health record system. In some implementations, the data translation component 1320 may translate patient health data into languages such as Spanish, Mandarin, or French to accommodate diverse patient populations.

In some implementations, the data translation component 1320 may normalize translated data to align with clinical terminology standards such as SNOMED CT, LOINC, or ICD-10. In some implementations, the data translation component 1320 may determine the correct clinical terminology by cross-referencing the translated data with a predefined ontology database. In some implementations, the data translation component 1320 may format the normalized data into structured fields compatible with HL7 or FHIR protocols for seamless integration into electronic health record systems. In some examples, the wearable data integration component 1322 may be configured as or otherwise support a means for integrating supplemental physiological data from wearable devices, including activity levels, sleep patterns, or heart rate trends, into the structured format for enhanced triage decision-making. In some implementations, the wearable data integration component 1322 may determine step count data from fitness trackers to assess daily activity levels. In some implementations, the wearable data integration component 1322 may interpret sleep duration and quality metrics from smartwatches to identify irregular sleep patterns.

In some implementations, the wearable data integration component 1322 may determine resting heart rate trends from wearable devices to identify potential deviations from baseline health metrics. In some implementations, the wearable data integration component 1322 may integrate hydration status data from wearable devices equipped with bioimpedance sensors to supplement physiological profiles. In some implementations, the wearable data integration component 1322 may determine stress levels by analyzing heart rate variability data collected from wearable devices during specific time intervals.

In some examples, the anomaly alert transmission component 1324 may be configured as or otherwise support a means for transmitting alerts to clinical systems in response to anomalies that may be indicative of acute physiological or neurological states exceeding predefined thresholds or dynamic thresholds unique to each person (i.e., personalization of thresholds). In some implementations, the anomaly alert transmission component 1324 may transmit alerts to electronic health record systems to notify providers of detected anomalies such as elevated heart rates or abnormal oxygen saturation levels. In some implementations, the anomaly alert transmission component 1324 may determine the appropriate clinical system for alert transmission based on the type of detected anomaly, such as respiratory distress or irregular blood pressure readings. In some implementations, the anomaly alert transmission component 1324 may transmit alerts to triage dashboards that rank patients based on the severity of detected anomalies, such as sudden changes in body temperature or irregular heart rhythms. In other embodiments, for example, deviations from personalized thresholds may trigger notifications to athletes and their coaches or trainers.

FIG. 14 shows a diagram of a system 1400 including a device 1402 that supports AI-guided clinical intake with biometric sensors and real-time data validation in accordance with aspects of the present disclosure. The device 1402 may be an example of or include the components of a database server or an apparatus 402 as described herein. The device 1402 may include components for bi-directional data communications including components for transmitting and receiving communications, including an AI-guided clinical intake component 1404, an I/O controller 1406, a database controller 1408, memory 1410, a processor 1412, and a database 1414. These components may be in electronic communication via one or more buses (e.g., bus 1416).

The AI-guided clinical intake component 1404 may be an example of an AI-guided clinical intake component 1206 or 1302 as described herein. For example, the AI-guided clinical intake component 1404 may perform any of the methods or processes described above with reference to FIGS. 12 and 13. In some cases, the AI-guided clinical intake component 1404 may be implemented in hardware, software executed by a processor, firmware, or any combination thereof.

The I/O controller 1406 may manage input signals 1418 and output signals 1420 for the device 1402. The I/O controller 14014 may also manage peripherals not integrated into the device 1402. In some cases, the I/O controller 14014 may represent a physical connection or port to an external peripheral. In some cases, the I/O controller 1406 may utilize an operating system such as iOS®, ANDROID®, MS-DOS®, MS-WINDOWS®, OS/2®, UNIX®, LINUX®, or another known operating system. In other cases, the I/O controller 1406 may represent or interact with a modem, a keyboard, a mouse, a touchscreen, or a similar device. In some cases, the I/O controller 1406 may be implemented as part of a processor. In some cases, a user may interact with the device 1402 via the I/O controller 1406 or via hardware components controlled by the I/O controller 1406.

The database controller 1408 may manage data storage and processing in a database 1414. In some cases, a user may interact with the database controller 1408. In other cases, the database controller 1408 may operate automatically without user interaction. The database 1414 may be an example of a single database, a distributed database, multiple distributed databases, a data store, a data lake, or an emergency backup database.

Memory 1410 may include random-access memory (RAM) and read-only memory (ROM). The memory 1410 may store computer-readable, computer-executable software including instructions that, when executed, cause the processor to perform various functions described herein. In some cases, the memory 1410 may contain, among other things, a basic input/output system (BIOS) which may control basic hardware or software operation such as the interaction with peripheral components or devices.

Processor 1412 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, the processor 1412 may be configured to operate a memory array using a memory controller. In other cases, a memory controller may be integrated into the processor 1412. The processor 1412 may be configured to execute computer-readable instructions stored in a memory 1410 to perform various functions (e.g., functions or tasks supporting AI-guided clinical intake with biometric sensors and real-time data contextualization).

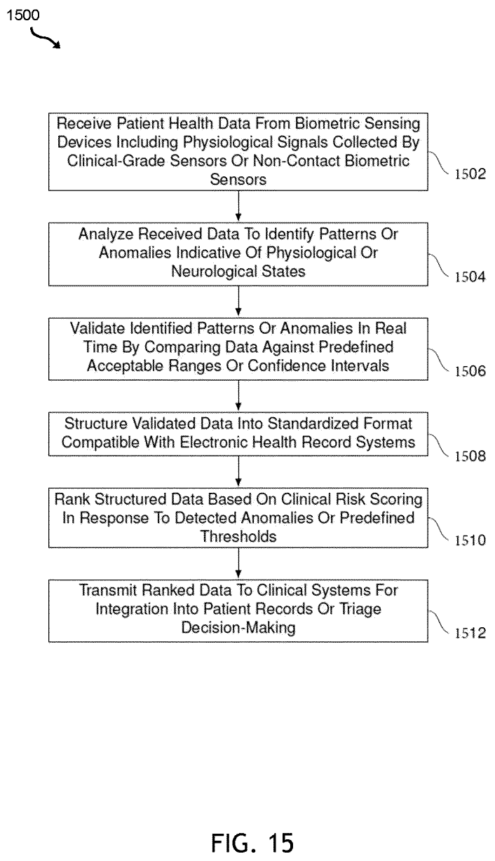

FIG. 15 shows a flowchart illustrating a method 1500 that supports AI-guided clinical intake with biometric sensors and real-time data validation in accordance with various aspects of the present disclosure. The operations of the method 1500 may be implemented by one or more components of a networked computing system as described herein. For example, the operations of the method 1500 may be performed by an AI-guided clinical intake component as described with reference to FIGS. 12 through 14. In some examples, one or more components of a networked computing system may execute a set of instructions to control the functional elements of the component(s) to perform the described functions. Additionally, or alternatively, the one or more components of a networked computing system may perform aspects of the described functions using special-purpose hardware.

At 1502, the method 1500 may include receiving patient health data from biometric sensing devices, the data including physiological signals collected by clinical-grade sensors or non-contact biometric sensors. The operations of 1502 may be performed in accordance with examples as disclosed herein. In some examples, aspects of the operations of 1502 may be performed by a data receiving component 504 as described with reference to FIG. 13.

At 1504, the method 1500 may include analyzing the received data to identify patterns or anomalies indicative of physiological or neurological states. The operations of 1504 may be performed in accordance with examples as disclosed herein. In some examples, aspects of the operations of 1504 may be performed by a pattern analysis component 1306 as described with reference to FIG. 13.

At 1506, method 1500 may include validating the identified patterns or anomalies in real time by comparing the data against predefined acceptable ranges or confidence intervals. The operations of 1506 may be performed in accordance with examples as disclosed herein. In some examples, aspects of the operations of 1506 may be performed by a real-time validation component 508 as described with reference to FIG. 13.

At 708, the method 1500 may include structuring the validated data into a standardized format compatible with electronic health record systems. The operations of 1508 may be performed in accordance with examples as disclosed herein. In some examples, aspects of the operations of 1508 may be performed by a data structuring component 1310 as described with reference to FIG. 13.

At 1510, the method 1500 may include ranking the structured data based on clinical risk scoring in response to detected anomalies, or dynamic or predefined thresholds. The operations of 1510 may be performed in accordance with examples as disclosed herein. In some examples, aspects of the operations of 1510 may be performed by a risk scoring component 1312 as described with reference to FIG. 13.

At 1512, the method 1500 may include transmitting the ranked data to clinical systems for integration into patient records or triage decision-making. The operations of 1512 may be performed in accordance with examples as disclosed herein. In some examples, aspects of the operations of 1512 may be performed by a data transmission component 1314 as described with reference to FIG. 13.

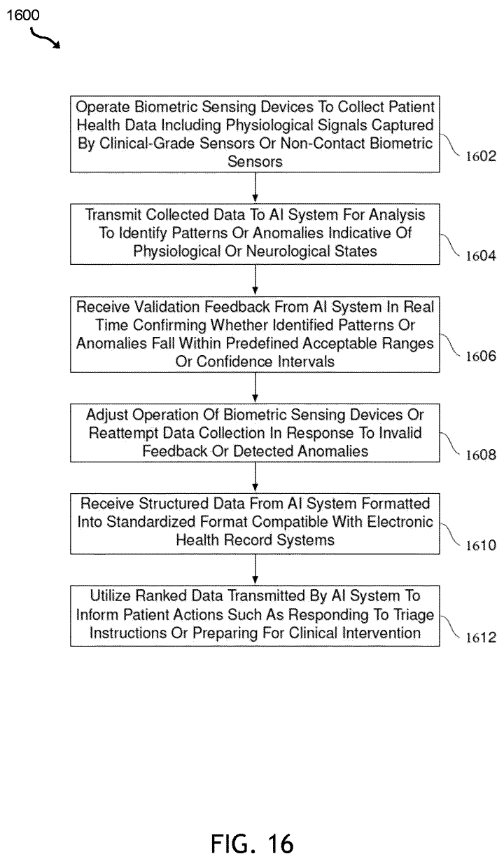

FIG. 16 shows a flowchart illustrating a method 1600 that supports AI-guided clinical intake with biometric sensors and real-time data validation in accordance with various aspects of the present disclosure. The operations of the method 1600 may be implemented by one or more components of a networked computing system as described herein. For example, the operations of the method 1600 may be performed by an AI-guided clinical intake component as described with reference to FIGS. 12 through 14. In some examples, one or more components of a networked computing system may execute a set of instructions to control the functional elements of the component(s) to perform the described functions. Additionally, or alternatively, the one or more components of a networked computing system may perform aspects of the described functions using special-purpose hardware.

At 1602, the method 1600 may include operating biometric sensing devices to collect patient health data, the data including physiological signals captured by clinical-grade sensors or non-contact biometric sensors. The operations of 1602 may be performed in accordance with examples as disclosed herein. In some examples, aspects of the operations of 1602 may be performed by a wearable data integration component 1322 as described with reference to FIG. 13.

At 1604, the method 1600 may include transmitting the collected data to an AI system for analysis to identify patterns or anomalies indicative of physiological or neurological states. The operations of 1604 may be performed in accordance with examples as disclosed herein. In some examples, aspects of the operations of 1604 may be performed by a data transmission component 1314 as described with reference to FIG. 13.

At 1606, the method 1600 may include receiving validated feedback from the AI system in real time, the feedback confirming whether the identified patterns or anomalies fall within predefined acceptable ranges or confidence intervals. The operations of 1606 may be performed in accordance with examples as disclosed herein. In some examples, aspects of the operations of 1606 may be performed by a real-time validation component 1308 as described with reference to FIG. 13.

At 1608, the method 1600 may include adjusting the operation of the biometric sensing devices or reattempting data collection in response to invalid feedback or detected anomalies. The operations of 1608 may be performed in accordance with examples as disclosed herein. In some examples, aspects of the operations of 1608 may be performed by an adaptive prompt generation component 1316 as described with reference to FIG. 13.

At 1610, the method 1600 may include receiving structured data from the AI system, the data formatted into a standardized format compatible with electronic health record systems. The operations of 1610 may be performed in accordance with examples as disclosed herein. In some examples, aspects of the operations of 1610 may be performed by a data structuring component 1310 as described with reference to FIG. 13.

At 1612, the method 1600 may include utilizing the ranked data transmitted by the AI system to inform patient actions, such as responding to triage instructions or preparing for clinical intervention. The operations of 1612 may be performed in accordance with examples as disclosed herein. In some examples, aspects of the operations of 1612 may be performed by a risk scoring component 1312 as described with reference to FIG. 13.

It should be noted that methods and systems described herein describe possible implementations, and that the operations and the steps may be rearranged or otherwise modified and that other implementations are possible. Furthermore, aspects of two or more of the implementations may be combined.

A number of alternative or optional embodiment aspects (“Aspects”) are described now to further illustrate the adaptability of the system(s) of the present invention. In Aspect 1, a method for AI-guided clinical intake with biometric sensors and real-time data validation, comprising: receiving patient health data from biometric sensing devices, the data including physiological signals collected by clinical-grade sensors or non-contact biometric sensors; analyzing the received data to identify patterns or anomalies indicative of physiological or neurological states; validating the identified patterns or anomalies in real time by comparing the data against predefined acceptable ranges or confidence intervals; structuring the validated data into a standardized format compatible with electronic health record systems; ranking the structured data based on clinical risk scoring in response to detected anomalies or predefined thresholds; and transmitting the ranked data to clinical systems for integration into patient records or triage decision-making. Aspect 2 furthers the method of aspect 1, comprising generating adaptive prompts to guide a patient through corrective actions in response to invalid physiological signals detected during real-time contextualization. Aspect 3 may comprise the method of any of Aspects 1 through 2, further comprising extracting emotional state indicators from voice tone, facial expressions, or biometric variations to supplement the ranking of structured data based on clinical risk scoring.

Aspect 4 comprises the method of any Aspects 1 through 3, further comprising translating patient health data into multiple languages and normalizing the translated data to clinical terminology standards compatible with electronic health record systems. Aspect 5 provides the method of any of Aspects 1 through 4, further comprising supplemental physiological data integrated from wearable devices, including activity levels, sleep patterns, or heart rate trends, into the structured format for enhanced triage decision-making.

Aspect 6 comprises the method of any of aspects 1 through 5, further comprising transmitting alerts to clinical systems in response to anomalies indicative of acute physiological or neurological states exceeding predefined thresholds. Aspect 7 comprises the method of any of aspects 1 through 6, wherein the structured data includes timestamps for each validated physiological signal to enable chronological tracking of patient health trends across multiple clinical encounters. Aspect 8 comprises the method of any aspects 1 through 7, wherein the contextualization engine applies confidence metrics derived from historical patient data to refine the acceptable ranges for physiological signals.

Aspect 9 comprises the method of any of Aspects 1 through 8, wherein the biometric sensing devices include multi-parameter sensors capable of simultaneously capturing blood pressure, heart rate, respiratory rate, and body temperature from a single anatomical location. Aspect 10 comprises the method of any of Aspects 1 through 9, wherein the transmitted data includes contextual metadata describing environmental conditions during signal collection, including ambient temperature, lighting, or noise levels.

Aspect 11 comprises a system for AI-guided clinical intake with biometric sensors and real-time data validation, comprising a processor; memory coupled with the processor; and instructions stored in the memory and executable by the processor to cause the system to perform a method of any of Aspects 1 through 10. Aspect 12 comprises a system for AI-guided clinical intake with biometric sensors and real-time data validation, comprising at least one means for performing a method of any of Aspects 1 through 10.

Aspect 13 comprises a non-transitory computer-readable medium storing code for AI-guided clinical intake with biometric sensors and real-time data validation, the code comprising instructions executable by a processor to perform a method of any of Aspects 1 through 10.

As already extensively described herein, there are numerous variations in the structure, configuration, communication, connectivity, distribution and utilization of the applications, systems, elements, and methods of the present invention. Although a wide variety of embodiments, constructs, elements, processes, and operations have been described above in connection with the present disclosure, those of skill in the art will appreciate that the above-described embodiments are merely examples of numerous embodiment of the present invention.

All embodiments described herein are presented for purposes of illustration and explanation only. The specific compositions, configurations, structures, processes, arrangements, and operations of various features and elements may be provided in a number of ways in accordance with the present disclosure and fully comprehended thereby.

Therefore, the embodiments and examples set forth herein are presented to best explain the present disclosure and its practical application, and to thereby enable those skilled in the art to make and utilize the disclosure. As previously explained, those skilled in the art will recognize that the foregoing description and examples have been presented for the purpose of illustration and example only. The disclosure as set forth is not intended to be exhaustive or to limit the disclosure to the precise form disclosed. Many modifications and variations are possible in light of the above teaching without departing from the spirit, scope, or enablement of the present disclosure.

Many modifications and variations are possible in light of the above teaching without departing from the spirit and scope of the present disclosure.

What is claimed is:

1. A digital system for evaluating one or more responses received from a subject of the system, the system comprising:
a database comprising subject-specific historical, functional, clinical, or operational data relevant to the subject;
a query engine that provides one or more queries, or adaptive guidance, for the subject;
a digital agent construct that interacts with the subject via the one or more queries, or adaptive guidance, from the query engine;
a sensor device that communicates or records a response characteristic of the subject during interaction with the digital agent construct;
an evaluation engine that analyzes unified data from a query, guidance, or response characteristic; wherein
the database; the query engine; the digital agent construct; the sensor device; and the evaluation engine are all communicatively or operationally intercoupled.

2. The system of claim 1, wherein the database further comprises a subject profile construct, communicatively or operationally coupled to each of the other constituent parts of the system, that provides historical, functional, clinical, or operational data from the subject.

3. The system of claim 1, wherein the database further comprises a reference profile construct, communicatively or operationally coupled to each of the other constituent parts of the system, that provides historical, functional, clinical, or operational data from a source other than the subject profile construct.

4. The system of claim 1, wherein the evaluation engine further comprises a pattern recognition module.

5. The system of claim 4, wherein the pattern recognition module further comprises a language understanding model.

6. The system of claim 1, wherein the digital agent construct comprises a machine learning construct.

7. The system of claim 1, wherein the digital system evaluates one or more clinical responses received from a human subject.

8. The system of claim 1, wherein the digital system evaluates one or more data responses received from a non-human subject.

9. The system of claim 1, wherein the evaluation engine, responsive to its evaluation of unified data, modifies further queries or adapative guidance provided by the query engine.

10. The system of claim 1, wherein the sensor device comprises a biometric sensor.

11. The system of claim 10, wherein the biometric sensor comprises a device worn by the subject.

12. The system of claim 1, wherein the sensor device comprises a biological sensor.

13. The system of claim 1, wherein the sensor device comprises an environmental sensor.

14. A digital clinical evaluation system for evaluating or more responses received from a human subject of the system, the system comprising:
a database storing a subject profile, comprising subject-specific historical, diagnostic, clinical, or observational data relevant to the subject;
a query engine that presents one or more queries for the subject;
a digital agent construct that interacts with the subject via a query;
a sensor device that communicates or records a response characteristic of the subject during interaction with the digital agent construct;
an evaluation engine that analyzes unified data from a query, guidance, or response characteristic; wherein
the database; the query engine; the digital agent construct; the sensor device; and the evaluation engine are all communicatively or operationally intercoupled.

15. The system of claim 14, wherein the database further comprises a reference profile construct, communicatively or operationally coupled to each of the other constituent parts of the system, that provides historical, diagnostic, clinical, or observational data from sources other than the subject profile.

16. The system of claim 14, wherein the evaluation engine further comprises a pattern recognition module.

17. The system of claim 14, wherein the pattern recognition module further comprises a language understanding model.

18. The system of claim 14, wherein the digital agent construct comprises a machine learning construct.

19. The system of claim 14, wherein the evolution engine, responsive to its evaluation of the unified data, modifies further queries.

20. A non-transitory machine readable medium comprising computer-executable instructions stored thereon, wherein the computer-executable instructions instruct one or more processors to perform a method comprising:
providing a database storing historical, functional, clinical, or operational data relevant to a subject;
providing a query engine that provides one or more queries, or adaptive guidance, for the subject;
providing a digital agent construct that interacts with the subject via the one or more queries, or adaptive guidance, from the query engine;
providing a sensor device that communicates or records a response characteristic of the subject during interaction with the digital agent construct;
providing an evaluation engine that analyzes unified data from a query, guidance, or response characteristic; wherein
the database; the query engine; the digital agent construct; the sensor device; and the evaluation engine are all communicatively or operationally intercoupled.
